(19) United States
(12) Patent Application Publication
(10) Pub. No.: US 2026/0108170 A1
(43) Pub. Date: Apr. 23, 2026

(54) PORTABLE RESPIRATION MONITORING SYSTEM POSITIONED IN THE PATHWAY OF RESPIRATION AND CONFIGURED WITH A DISASSEMBLEABLE SENSOR/ELECTRONIC ASSEMBLY WITH A DISPOSABLE THERMAL SENSOR

(71) Applicant: LINSHOM MEDICAL, INC., ELLICOTT CITY, MD (US)

(72) Inventors: JOHN MOSER, DOVER, DE (US); JAMES RICHARD HUGHEN, ANNAPOLIS, MD (US); URI FELDMAN, COLUMBIA, MD (US); DORON FELDMAN, WILLIAMSVILLE, NY (US); RONEN FELDMAN, ELLICOTT CITY, MD (US); DOUGLAS PEMBERTON, TIMONIUM, MD (US); KEVIN BARNES, BALTIMORE, MD (US)

(21) Appl. No.: 18/486,438

(22) Filed: Oct. 13, 2023

Related U.S. Application Data
(60) Provisional application No. 63/379,500, filed on Oct. 14, 2022.

(51) Int. Cl. A61B 5/08; A61B 5/00

(52) U.S. Cl. CPC A61B 5/0803; A61B 5/6803; A61B 2560/0406; A61B 2560/0443; A61B 2560/0462; A61B 2560/06; A61B 2562/0271; A61B 2562/166

(57) ABSTRACT

A portable respiration monitoring system usable wherever the respiratory monitoring is needed comprises a sensor/electronic assembly having disposable sensor components, non-disposable electronic components, and a positioning device for securing the sensor components in the respiration pathway of a subject. The positioning device is envisioned as a mask, nasal cannula, a nares clip, as hanging from eyeglasses, attached to the skin by an adhesive or magnetically in proximity to the nose or mouth, in a ET tube, in a tracheostomy tube, in a CPAP machine, in a Vapotherm/Fisher Panel device, etc. The disposable sensor and non-disposable electronic components are removably coupled with one another for a duration of the respiration monitoring, and de-coupled from one another when monitoring is completed, so that the disposable sensor components are disposed of, while the non-disposable electronic components may be re-used.

### REFERENCES TO RELATED PATENT APPLICATION(S)

This Utility Patent Application is based on a Provisional Patent Application Ser. No. 63/379,500, filed on 14 Oct. 2022.

### INCORPORATION BY REFERENCE

U.S. Pat. No. 8,911,380 filed on 19 Jul. 2012 and issued on Dec. 16, 2014, which is based on a Provisional Patent Application Ser. No. 61/170,594, filed on 17 Apr. 2009, is incorporated herein by reference in its entirety.

### FIELD OF THE INVENTION

The present invention relates to medical systems and methods, and particularly to a system and method for monitoring of the respiration of a subject, or a patient.

In particular, the present invention is directed to a sensor/electronic assembly system positioned in the pathway of respiration (or in proximity to the subject's nose or mouth) either by attachment to a facial mask/nasal cannula, or a nares clip, or hanging from eyeglasses, or attached to the skin by an adhesive or magnetically in proximity to the nose or mouth, or in a tracheostomy tube, or in a CPAP machine, or a Vapotherm/Fisher Panel device, etc. (further referred to herein intermittently as a mask) for monitoring the respiration of a subject, where the sensor/electronic assembly system is configured with a disposable sensor components and a non-disposable data processing components removably coupled/de-coupled with/from one another for a duration of the mask usage.

Furthermore, the present invention addresses a respiration monitoring system which uses a mask (or other devices including, but no limited to, nasal cannula, or a nares clip, or hanging from eyeglasses, or secured to the skin by an adhesive or magnetically in proximity to the nose or mouth, or in a tracheostomy tube, or in a CPAP machine, or a Vapotherm/Fisher Panel device, etc.) equipped with a disposable thermistor sub-system and a non-disposable electronic sub-system, where the disposable and non-disposable sub-systems of the present respiration monitoring system are coupled one to another through a reliable connection mechanism (also referred to herein as a locking and/or mating sub-system) which provides an easy assembling/disassembling of the disposable thermistor sub-system and the non-disposable electronic sub-system to/from one another, and wherein the disposable sensor sub-system along with the mask (or other device placeable in proximity to the mouth and/or mouth of a subject) can be disposed of upon the completion of usage, while the non-disposable electronic sub-system is not disposed of, but is saved and may be re-used with a different subject, or patient.

In addition, the present invention addresses a portable respiration monitoring system equipped with a sensor/electronic assembly built with sensor sub-assembly/electronic sub-assembly which are locked to one another and to the mask/cannula (or other device placeable in proximity to the mouth and/or mouth of a subject) through a locking/mating mechanism, and which can be disassembled from one another when the respiration monitoring is completed and/or the mask (or other device placeable in proximity to the mouth and/or mouth of a subject) is to be disposed of, where the sensor/electronic assembly includes a disposable thermistor portion (which can be disposed of along with the mask) and a non-disposable electronic portion which includes a printed circuit board (PCB), electrical connections from the thermistor to the PCB, a thermal electric cooler (TEC), a cable electrically coupled to the PCB, and where the non-disposable electronic portion may be re-used from a subject to subject, or patient to patient.

Further, the present invention pertains to a respiration system for monitoring the respiration of a patient, which is capable of generating alarm signals when respiration parameters of the patient deviate from a predetermined threshold.

In addition, the present invention relates to a respiration monitoring system configured for the measurement of a patient's breathing parameters substantially independent of the inference of conditions within the external environment.

The present invention also relates to a substantially portable respiration monitoring system that is particularly adaptable for use wherever the respiratory monitoring is needed, such as, for example, but not limited to the usage at home, or in pre-hospital (EMT, paramedic, ambulance) and hospital settings, or in transport for military applications, at consumer market settings (such as, for example, CVS, Walgreen, etc.), or in procedures performed in medical and/or dental offices, as well as under extreme conditions, where it is not possible to maintain viable contact with a subject in distress, or at sites of accidents, for miners, firemen, military personnel, etc.

### BACKGROUND OF THE INVENTION

Brain damage and/or death of a subject (or a patient) are direct consequences of prolonged apnea (absence of respiration), particularly, when combined with hypoxia. Diagnosing apnea and hypoventilation (reduction in breathing rate) are of particular concern when a patient is sedated, or when his/her breathing fails during sleep (as in obstructive sleep apnea).

Identifying hypoventilation before apnea occurs by monitoring breathing frequency and/or breath volume of the patient allows time to stimulate breathing, relieve airway obstruction, and restore effective respiration of the subject before hypoxia occurs. Hypoxic-ischemic brain damage may result from inadequate or loss of respiration, which, if not reversed early in the development, may lead to brain damage and possible death.

Generally, respiratory rate can be monitored continuously and non-invasively by measuring the carbon-dioxide partial pressure in the exhaled breath. However, carbon-dioxide measurement is not an accurate measure of TV.

Ventilation may also be measured by examining displacement of the chest and rib cage using sophisticated monitors found in operating room and in sleep laboratories. However, such monitors are not transportable, are cumbersome in use, generally expensive, and are not widely available.

At the present time, portable, disposable and inexpensive carbon-dioxide detectors are needed which would be able to provide for non-invasive measures of ventilation in a patient whose airways are not instrumented in some way. Monitoring systems are needed to detect the presence of breathing, to measure respiratory rate and to estimate the breathing volume in patient both in hospitals and outside hospital settings, in doctor's offices, at home, and in the community (for firemen, miners, armed forces, etc.).

In some prior art respiration monitoring systems, such as described in U.S. Pat. No. 5,069,222, the respiration monitoring includes the use of thermistors to develop an electrical signal representative of a patient's breath temperature. Other prior art systems for monitoring a patient's respiration (as exemplified by U.S. Pat. No. 5,190,048) provide for thermistors to be aligned with the nasal passages of a patient and mounted in an air flow sensor assembly. Also, respiration monitoring systems described in U.S. Pat. Nos. 6,165,133 and 6,272,933, utilize thermistor technology inserted or positioned in the vicinity of a patient's nasal passages.

Unfortunately, these prior art respiration monitoring systems have substantial shortcomings, such as, for example, not taking into account the effects of the ambient environment temperature effects on the electric signal. As the result, an erroneous monitoring status may be reported when the ambient temperature is similar to the breath temperature or where the ambient temperature is experiencing rapid changes in temperature. These systems also rely solely on the signal from the thermistors to monitor the patient's breath temperature, and are disadvantaged by lacking means to isolate the monitoring system from external changing parameters, or lacking any mechanism for solving the problem of changing ambient thermal parameters, or being able to obtain a reliable respiration signal when the ambient environment temperature is close to the breath temperature of the patient.

These prior art systems are also deficient in that they are either completely disposed of after usage, or should be completely sterilized, thus, disadvantageously, opposing the signal processing components to high temperatures associated with sterilization, which is undesirable in view of the expensiveness of electronic system used in such respiration monitoring systems.

U.S. Pat. No. 8,911,380, incorporated herein by reference in its entirety, describes a respiratory monitoring system which includes a thermal electric generator that may be mounted within a mask enclosure (or free-standing), covering all or part of the nose and/or mouth, of a subject (a patient). A first temperature sensor is attached to the thermoelectric generator for measuring the subject's breath. A power controller develops a difference between a preset temperature and the subject's breath temperature that is subsequently input into a feedback error signal and also into a power controller which regulates a power to the thermoelectric generator to maintain a preset temperature.

Although the respiration monitoring system described in U.S. Pat. No. 8,911,380 obviates the shortcomings of the prior art systems with regard to solving the problem of the changing ambient thermal parameters and is able to obtain a reliable respiration signal when the ambient environment temperature is close to the breath temperature of the patient, this respiration monitoring system however still needs to be improved. Specifically, the system described in U.S. Pat. No. 8,911,380 uses a single-piece sensor/electronic module, shown in FIG. 1, which includes a thermistor sensor in an integral electrical and mechanical coupling with the signal processing electronics. The one-piece sensor/electronic module may be located within a face mask, nose mask, or simply adjacent to the nostrils of the subject and is based on the electronics depicted in FIG. 2.

Such respiration monitoring system, although being able to provide a reliable respiration reading independent of the ambient environmental temperatures, still needs a further improvement as it is based on the single-piece sensor design which either should be disposed of in its entirety or be sterilized for re-usage. The single-piece design, thus, is associated with a high cost (as it requires to dispose of the expensive electronics), or it may suffer inaccuracies resulting from exposure of the electronics to high sterilization temperatures.

The respiration monitoring system which based on a single-piece sensor design disadvantageously does not permit disposal of the mask/nasal cannula along with the inexpensive parts of the sensor system (such as the thermistor) while keeping the expensive electronics of the system non-disposed for further re-usage.

It would be highly desirable, in lieu of the disadvantages of the prior art respiration monitoring systems to provide a sensor/electronic assembly for respiration monitoring systems which would be capable of keeping and reusing the expensive electronic components, and which would be capable of disposing of relatively inexpensive components of the sensor system.

### SUMMARY OF THE INVENTION

It is therefore an object of the present invention to provide a portable system for monitoring the respiration of a subject, which includes a disposable sensor sub-system which can be disposed of along with a device providing positioning of the disposable sensor sub-system in the pathway of respiration or in proximity to the nose and/or mouth (further referred to herein as a mask or a disposable mask) when the usage is completed, and a non-disposable electronic sub-system which can be kept and be re-used.

It is another object of the present invention to provide a portable respiration monitoring system which includes a disposable mask, a disposable sensor sub-system attachable to the disposable mask, and a non-disposable electronic sub-system which are interconnected through a coupling mechanism (also referred to herein as a locking sub-system or mating sub-system) which reliably locks the disposable and non-disposable sub-systems to one another for a period of the mask usage, and which can be disengaged to remove the expensive non-disposable electronic sub-system from the inexpensive disposable sub-system and the disposable mask when the usage of the system is completed and the disposable sensor sub-system, along with the mask, can be disposed of.

In one aspect, the present invention addresses a portable respiration monitoring system, which comprises a disposable mask affixable in a respiration pathway, and a sensor/electronic assembly attachable to the disposable mask and configured with
(a) a disposable sensor sub-assembly,
(b) a non-disposable electronic sub-assembly, and
(c) a locking sub-system operationally coupled to the disposable mask, the disposable sensor sub-assembly and the non-disposable electronic sub-assembly.

The locking sub-system is configured for intermittent coupling/decoupling of the non-disposable electronic sub-assembly to/from the disposable sensor sub-assembly and the disposable mask.

The disposable sensor sub-assembly (also referred to herein as the thermistor sensor sub-system) includes a thermistor element, a thermistor retainer configured to hold and secure the thermistor element, a pair of contact pins, and a pair of thermistor wires coupled between the thermistor element and the contact pins.

The locking sub-system includes a disposable sensor carrier which is configured to house the thermistor element, thermistor retainer, the thermistor wires, and the contact pins. The disposable mask is configured with a sensor cut-out formed in a wall of the mask. The disposable sensor carrier is attached to the disposable mask at the cut-out with the thermistor element positioned inside the disposable mask.

The disposable sensor carrier is attached to the disposable mask and snaps on the non-disposable electronic sub-sub-assembly to form the mechanical, as well as thermal and electrical, connection between the disposable sensor sub-assembly and non-disposable electronic sub-assembly for the duration of the respiration monitoring procedure. The disposable sensor carrier (along with the disposable sensor sub-assembly “stuffed” therein) is unplugged from the non-disposable electronic sub-assembly to terminate electrical, mechanical, and thermal connection therebetween, when the monitoring routine is completed.

The non-disposable electronic sub-assembly includes a non-disposable housing, which incorporates a Printed Circuit Board (PCB) sub-assembly populated with PCB electrical connections (also referred to herein as the PCB leads or PCB wires) formed thereon, a pair of spring contacts formed in an electrical contact with said PCB electrical connections, a thermal electric cooler (TEC), a pair of TEC wires coupled to the TEC and to the PCB electrical connections, and a heat sink in thermal contact with the TEC.

When prepared for the usage, the disposable sensor carrier (“stuffed” with the components of the thermistor sensor sub-system) is attached to the mask, and the non-disposable housing (“stuffed” with the components of the non-disposable electronic sub-assembly) is attached to the disposable sensor carrier, resulting in bringing thermistor wires into electrical contact with the PCB leads via the spring contacts of the non-disposable electronic sub-assembly and the contact pins of the disposable sensor sub-assembly.

Preferably, the contact pins of the disposable sensor sub-assembly are configured as POGO pins, each inserted in a respective indentation formed on the disposable sensor carrier.

The thermistor retainer has a retainer body configured with a retainer top portion carrying the thermistor element, a retainer bottom portion brought in a contact with the TEC of the non-disposable electronic sub-assembly (when assembled), and a retainer channel extending through the retainer body between its top and bottom portions. The pair of thermistor wires extend within the retainer channel from the thermistor element out of the retainer body in contact with the POGO pins, and further either are pressed down by the POGO pins to the PCB wires, or the contact between the thermistor wires and the PCB wires is obtained through the POGO pins.

The non-disposable electronic sub-assembly further comprises a sensor cable having a wire sub-system extending therethrough. The wire sub-system within the sensor cable is coupled to the non-disposable housing in an electrical contact with the PCB wires, as well as the TEC wires and the thermistor wires through the PCB wires.

The non-disposable housing of the non-disposable electronic sub-system includes a front portion, a rear portion attached to the front portion, a gasket member secured between the front and rear portions, and a cable entrance opening formed in a bottom wall of the non-disposable housing for passing one end of the sensor cable therethrough to bring the wire system of the sensor cable in electrical contact with the PCB sub-system.

The disposable sensor carrier preferably includes two parallel plates displaced from one another to secure a wall of the disposable mask therebetween.

The locking sub-system is further configured with a disposable sensor carrier/non-disposable housing interface, which may be formed with bottom tabs positioned at a bottom of the disposable sensor carrier, wherein the front portion of the non-disposable housing is configured with a front wall having a front opening, and wherein the bottom tabs of the sensor carrier cooperate with the front wall of the front portion of the non-disposable housing within the front opening to connect the disposable sensor carrier and the non-disposable housing to one another. The rear portion of the non-disposable housing may be secured to the front portion of the non-disposable housing via a system of connecting pins, or via adhesive. When the subject system is assembled, the spring contacts of the non-disposable electronic sub-assembly extend within the front opening of the front portion of the non-disposable housing in electrical contact with the POGO pins and the thermistor wires of the disposable sensor sub-system.

The PCB sub-assembly is disposed in the rear portion of the non-disposable housing. The non-disposable housing further includes a compression spring disposed at top portion thereof to maintain the PCB sub-assembly at a predetermined position within the non-disposable housing. The non-disposable housing further includes a compression spring latch positioned in proximity to the front portion of the non-disposable housing and securing the compression ring in place.

The subject system further comprises a package which includes a plurality of non-disposable electronic sub-assemblies packaged in a first corrugated box, with each non-disposable electronic sub-assembly positioned in a separate plastic bag, and a plurality of disposable sensor sub-assemblies packaged in a second corrugated box, with each disposable sensor sub-assembly and the disposable mask packaged in a separate plastic bag.

In another aspect, the present invention addresses a method for monitoring respiration function of a subject, which includes the following steps:
configuring a disposable sensor sub-system of a respiration monitoring system with a disposable sensor carrier incorporated with a thermistor element, a pair of thermistor wires, and a pair of POGO pins in an electrical contact with the pair of thermistor wires,
attaching the disposable sensor sub-system to a disposable mask,
configuring a non-disposable electronic sub-system of the respiration monitoring system with a non-disposable housing incorporated with a Printed Circuit Board (PCB) sub-assembly populated with PCB wires formed thereon, a pair of spring contacts formed in an electrical contact with said PCB wires, a thermal electric cooler (TEC), a pair of TEC wires coupled to the TEC and to the PCB electrical connections, a heat sink in thermal contact with the TEC, and a sensor cable electrically coupled to the PCB wires,
operating the respiration monitoring system intermittently in a monitoring mode of operation and in a between-monitoring mode of operation,
coupling said non-disposable housing of said non-disposable electronic sub-system to said disposable sensor carrier of said disposable sensor sub-system, thus connecting said disposable sensor sub-system and said non-disposable electronic sub-system for the monitoring mode of operation,
decoupling said non-disposable housing of said non-disposable electronic sub-system from said disposable sensor carrier of said disposable sensor sub-system, thus disconnecting said non-disposable electronic sub-system from said disposable sensor sub-system for the between-monitoring mode of operation, and
disposing of said disposable mask and disposable sensor sub-assembly while keeping the non-disposable electronic sub-system.

These and other objects of the present invention will be more apparent after reading the further Description of the Preferred Embodiment(s) in conjunction with the accompanying Patent Drawings in the current Application.

### BRIEF DESCRIPTION OF THE DRAWINGS

FIG. 1 is a single-piece sensor design for a prior art respiratory monitoring system;

FIG. 2 is a block diagram of the control loop of the hardware disclosed in the U.S. Pat. No. 8,911,380 which is fully applicable with respect to the subject monitoring system;

FIGS. 3A-3C are representative of an exemplary embodiment of the subject respiratory monitoring system;

FIG. 4 is a simplified illustration of the subject two-piece sensor assembly having an internal disposable thermistor sub-system, and an external non-disposable electronic sub-system;

FIG. 5 is a simplified view of the components of the subject disposable thermistor sub-system;

FIG. 6 is a simplified representation of the components of the subject non-disposable electronic sub-system;

FIG. 7 is a simplified illustration of the subject sensor assembly showing the mated disposable thermistor sub-system and non-disposable electronic sub-system attached to the disposable mask;

FIGS. 8A-8C depict an exemplary embodiment of the subject sensor module with FIG. 8A being a side view of the disposable and non-disposable sub-systems, FIG. 8B being an exploded view of the subject sensor module, and FIG. 8C being a perspective view of the subject sensor module;

FIG. 9A is a top view of a surface mounted thermistor;

FIG. 9B is a detailed side view of the surface mounted thermistor shown in FIG. 9A;

FIG. 10 shows a wafer with thermistors (for the surface mounted embodiment shown in FIG. 9A-9B) loaded for a reflow process;

FIG. 11A-11E are representative of a module assembly in the subject non-disposable electronic sub-system, where FIG. 11A is the perspective view of the subject module assembly (exposing a front portion of the non-disposable housing) connected with the sensor cable, FIG. 11B is a perspective view of the subject module assembly (exposing a rear portion of the non-disposable housing) in connection with the sensor cable, FIG. 11C is a side view of FIG. 11A-11B, FIG. 11D is a front view of the subject module assembly of FIG. 11A, also showing the top and bottom views of the non-disposable housing of the subject module assembly, and FIG. 11D showing a longitudinal cross-section of the subject module assembly taken along Lines A-A of FIG. 11A;

FIG. 12A is a pictorial view of the subject non-disposable housing showing the disposable sensor carrier attached thereto,

FIG. 12B is a cross-section taken along Lines A-A of FIG. 12A and presenting details of the subject sensor assembly;

FIGS. 13A and 13B are side and front pictorial views, respectively, of the subject disposable sensor carrier attached to the mask and to the non-disposable housing;

FIG. 14A is a perspective view of the disposable sensor carrier attached to the front of the non-disposable housing in correspondence to FIG. 13A,

FIG. 14B is a cross-section of FIG. 14A taken along Lines A-A;

FIG. 15A-15B are pictorial views of the disposable sensor carrier shown from the front (FIG. 15A) and rear (FIG. 15B);

FIG. 15C is a cross-section of the disposable sensor carrier taken along Lines A-A of FIG. 15A;

FIG. 15D is a cross-section of the disposable sensor carrier “stuffed” with the disposable sensor components taken along Lines B-B of FIG. 14A;

FIG. 16A shows the sensor cable assembly of the subject system;

FIG. 16B shows the front and rear views of the entrance of the cable wires into the connection header housing;

FIG. 16C details the interconnection between the sensor cable and the connection header housing;

FIG. 16D is a side view of the PCB assembly with the connection header housing attached to the bottom of the non-disposable PCB and the non-disposable components (TEC, spring contacts) attached to the top of the PCB;

FIGS. 16E and 16F are pictorial views of the top of the PCB (with FIG. 16E showing one side of the PCB and FIG. 16F showing another side of the PCB);

FIG. 16G is a side view of the PCB assembly;

FIG. 17A-17B depict the front portion of the non-disposable housing, with FIG. 17A being the pictorial view of the front portion, and, FIG. 17B being a rear internal view of FIG. 17A;

FIG. 18A-18D are respectively cross-sections of the non-disposable housing, with FIG. 18A showing a cross-section taken along Lines A-A of FIG. 17B, FIG. 18B showing a cross-section taken along Lines B-B of FIG. 17B, FIG. 18C being a cross-section taken along Lines C-C of FIG. 17A, and FIG. 18D being an enlarged portion D of FIG. 18C;

FIG. 19A-19D detail the structural elements of the rear portion of the non-disposable housing of the subject sensor module assembly with FIG. 19A being a pictorial view of the rear portion, FIG. 19B showing a cross-section taken along Lines B-B of FIG. 19A, FIG. 19C being a cross-section taken along Lines A-A of FIG. 19A, and FIG. 19D being a side view of the rear portion of FIG. 19A;

FIG. 20 is a pictorial view of the gasket in the non-disposable housing;

FIG. 21A is a pictorial view of the compression spring latch insertable at the front portion of the non-disposable housing of the subject sensor module, and FIG. 21B is the cross-section taken along Lines A-A of FIG. 21A;

FIG. 22A-22C detail the thermistor retainer with FIG. 22A being a perspective view showing the bottom of the thermistor retainer, FIG. 22B being a perspective view showing the top of the thermistor retainer, and FIG. 22C showing the longitudinal cross-section of FIG. 22A-22B;

FIG. 23 shows the thermistor element used in the subject system;

FIG. 24 shows the non-disposable electronic package; and

FIG. 25 shows the disposable sensor/mask package.

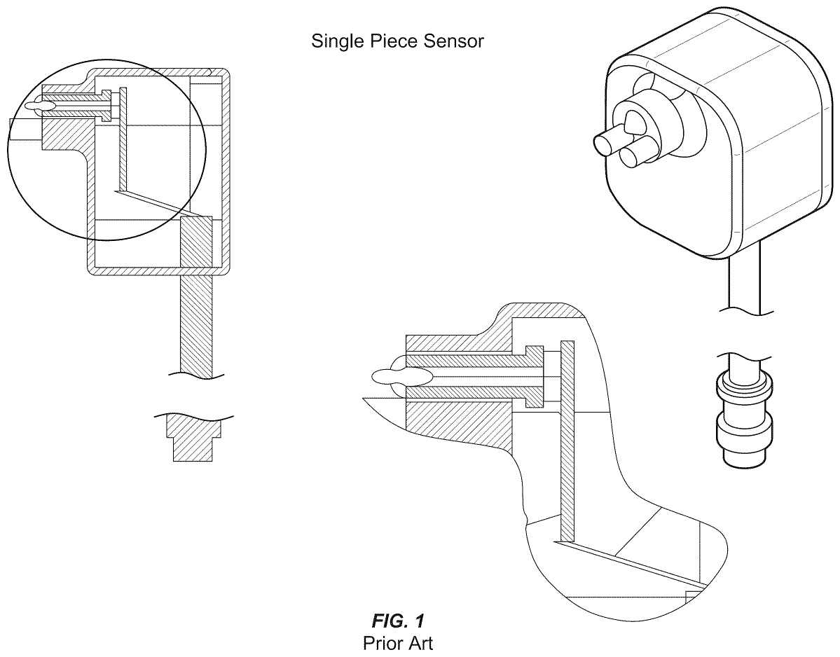

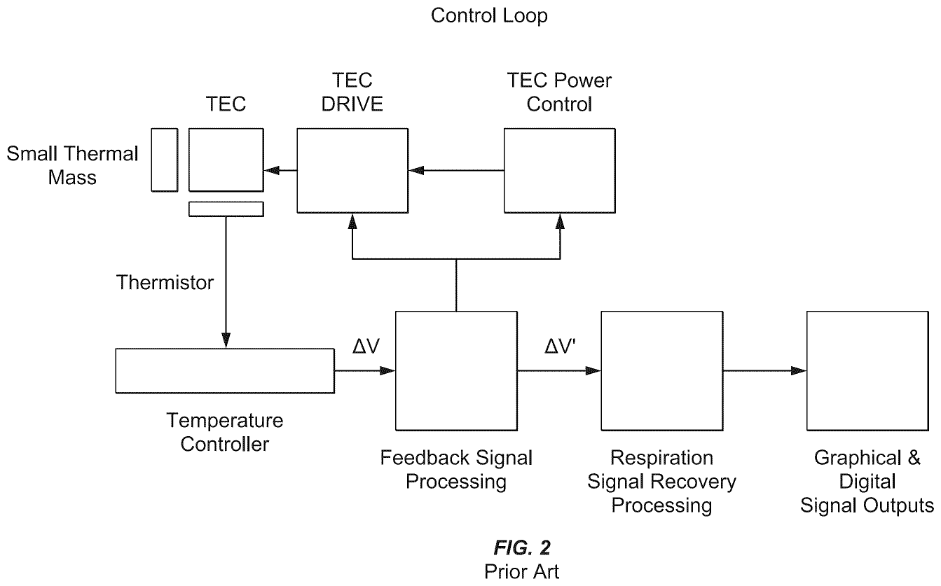

### DETAILED DESCRIPTION OF THE PREFERRED EMBODIMENT(S)

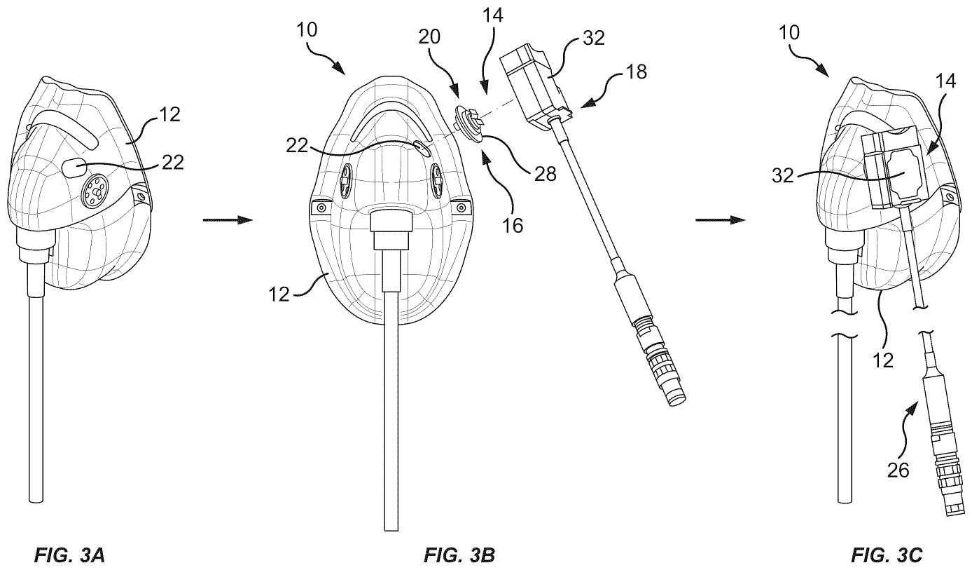

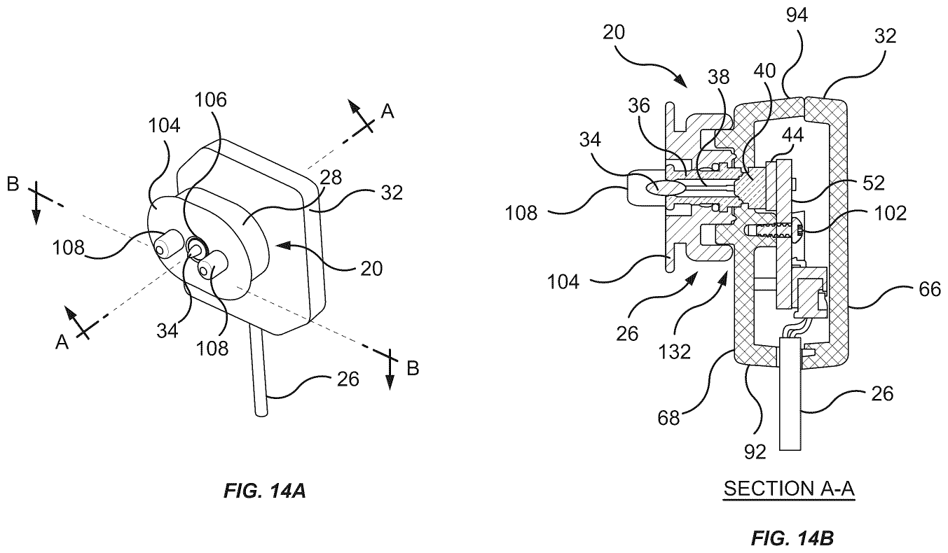

FIGS. 3A-25 depict the subject respiration monitoring system 10 for monitoring the respiration of a subject (also referred to herein as a patient). The system 10 is the sensor/electronic sub-assembly having a sensor sub-assembly and an electronic sub-assembly. The sensor sub-assembly may be affixed, for the duration of the respiratory monitoring, in the pathway of respiration of the subject by a device providing positioning of the sensor sub-assembly in proximity to the mouth or nose of the subject. Such device is envisioned in numerous forms. For example, the positioning of sensor sub-assembly in the respiration pathway may be either by attachment of the system 10 to a facial mask/nasal cannula, or a nares clip, or hanging from eyeglasses, or attached to the skin by an adhesive or magnetically in proximity to the nose or mouth, or in a tracheostomy tube, or in a CPAP machine, or a Vapotherm/Fisher Panel device, etc. (further referred to herein intermittently as a mask or a disposable mask 12) for monitoring the respiration of a subject. The mask 12 (or any other device) may be fastened to the subject by flexible straps or some other fastening means. When using the mask for the respiration monitoring, such device may be adapted to be held against the face (or a nose or a mouth) of a subject to ensure that the most of the subject's breathing passes through the mask 12. Although applicable to any arrangement for affixing the system 10 in the respiration pathway, such will be further referred to herein as the mask (or disposable mask), and, only as an example, but not to limit the scope of the present invention, the present system 10 will be described herein in association with a disposable mask 12.

The respiration monitoring system 10 is highly portable system that may be adaptable for use wherever the respiratory monitoring is needed, such as, for example, but not limited to the usage at home, or in pre-hospital (EMT, paramedic, ambulance) and hospital settings, or in transport for military applications, at consumer market settings (such as, for example, CVS, Walgreen, etc.), or in procedures performed in medical and/or dental offices, as well as under extreme conditions, where it is not possible to maintain viable contact with a subject in distress, or at sites of accidents, for miners, firemen, military personnel, etc.

The present system is envisioned for usage in operating rooms, but may also be adapted for use at external sites that do not have the full capabilities and instrumentation of a hospital operating room for the respiration of a subject. The subject system 10 may be used in different environments encountered by medical personnel where the system 10 can be easily transported from one area to another in a short amount of time with a minimal effort, or outside healthcare systems, such as by ambulances, and worn by firemen, miners, etc.

The respiration monitoring system 10 is adapted to be used in varying external ambient conditions which may provide for ambient external temperatures covering a wide temperature range and possibly subject to rapid ambient temperature changes. In order to provide accurate readings of the object's status, independent of the external ambient temperatures, the subject system 10 may be provided with a feedback loop electronics which is presented in U.S. Pat. No. 8,911,380 incorporated herein in its entirety, and applicable to the present system 10 with the exception that the temperature sensor in the present system 10 is a disposable module which can be secured to the electronic sub-system for the duration of the usage and disassemble from the electronic sub-system when the respiration monitoring has been completed. Upon the respiration monitoring has been completed, the sensor module along with the mask can be disposed of, while the electronic sub-system can be kept to re-usage with another mask for respiration monitoring of another subject.

Although being envisioned for usage with various devices for affixing the subject portable respiration monitoring system 10 in the pathway if respiration, as one of numerous embodiments, an exemplary implementation of the present portable respiration monitoring system 10 uses a disposable mask 12, as specifically depicted in FIG. 3A-3C. However, any type of a facial attachment applicable for monitoring the respiration is also contemplated for usage with the subject system 10 which includes a sensor/electronic assembly 14 which is configured with a disposable sensor sub-assembly 16 and non-disposable electronic sub-assembly 18. The disposable sensor sub-assembly 16 is attached to the mask 12, while the non-disposable electronic sub-assembly 18 is attached to the mask 12 and to the disposable sensor sub-assembly 16 in a removable fashion through a connecting mechanism (also referred to herein as a locking sub-system or a mating sub-system) 20 which provides a reliable connection between the disposable and non-disposable sub-assemblies 16 and 18 and easy removal from one another.

As shown in FIG. 3A-3B, the mask 12 is configured with a cutout 22 formed in the wall 30 of the mask body. As shown, cutout 22 is configured as an oval contoured opening dimensioned and formed in a configuration cooperating with an oval shaped disposable sensor carrier 28 of the connecting mechanism 20. It is to be understood, however, that the cutout 22 can alternatively be contoured also in any other configuration which would cooperate with the contour of the sensor carrier 28 if contoured in a different shape.

The non-disposable electronic sub-assembly 18 in the subject system 10 includes electronic components including a PCB sub-assembly, thermal electric cooler (TEC) heat sink, etc., which, as will be detailed in further paragraphs, represent an expensive portion of the subject respiration monitoring system 10 and which can be detached from the disposable temperature sensor sub-assembly 16 and from the mask 12 and re-usable from a patient to another patient. The disposable sensor sub-assembly 16, however, being a relatively inexpensive part of the present system 10, is attached to the mask 12 and can be disposed of along with the mask when its usage is completed. Generally, the disposable sensor sub-assembly 16 may include a disposable thermistor element 34 which is disposable along with the mask 12 after the respiration monitoring for a subject has been completed.

As shown in FIG. 3A-3C, in assembling the present portable respiration monitoring system 10, a mask 12 (or a nasal cannula, or any other device capable of being secured to the face, mouth or nose of a subject) is assembled with the disposable sensor sub-assembly 16 (detailed in further paragraphs) as shown in FIG. 3B, and the non-disposable electronic sub-assembly 18 is subsequently attached to the mask 12 with the non-disposable housing module 32 (which contains the non-disposable components) secured to the mask 12 by means of the disposable sensor carrier 28 (which contains the disposable components), as will be detailed in further paragraphs.

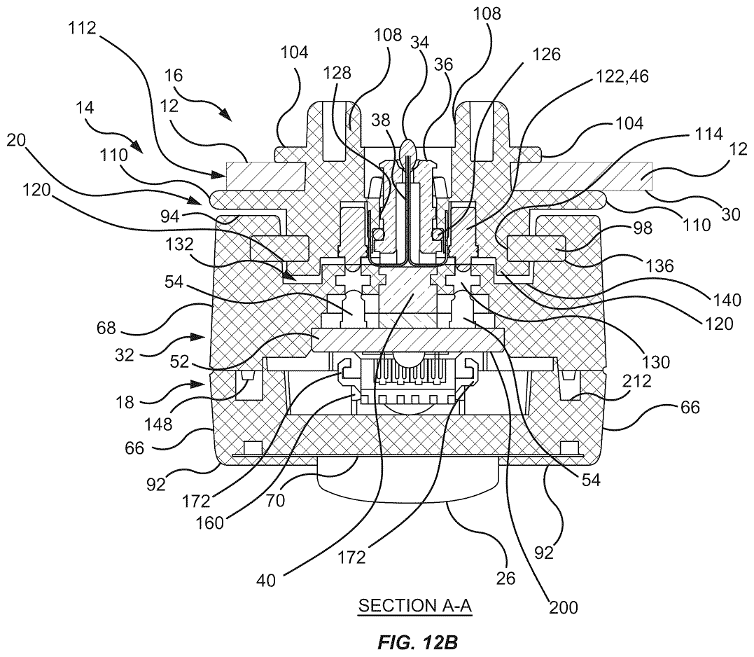

The sensor carrier 28 constitutes a part of the locking sub-system 20 as it participates in interlocking the non-disposable electronic sub-assembly 18 with the disposable sensor sub-assembly 16 and the disposable mask 12. Referring to FIG. 3B-3C, the non-disposable sub-assembly 18 includes the non-disposable housing module 32, the sensor cable 26, and is removably assembled with the disposable sensor carrier 28, where the sensor carrier 28 represents a portion of the connecting mechanism 20, as best presented in FIGS. 12A-12B, 13A-13B, 14A-14B, and 15A-15D, which detail the manner in which the disposable sensor sub-assembly 16 and non-disposable electronic sub-assembly 18 interface one another. The disposable sensor carrier 28, being attached to the wall 30 of the mask 12 within the cut-out 22, holds the disposable components in electrical/thermal/mechanical engagement with the non-disposable electronics in the non-disposable housing module 32 with the sensor cable 26 in place during the respiration monitoring cycle.

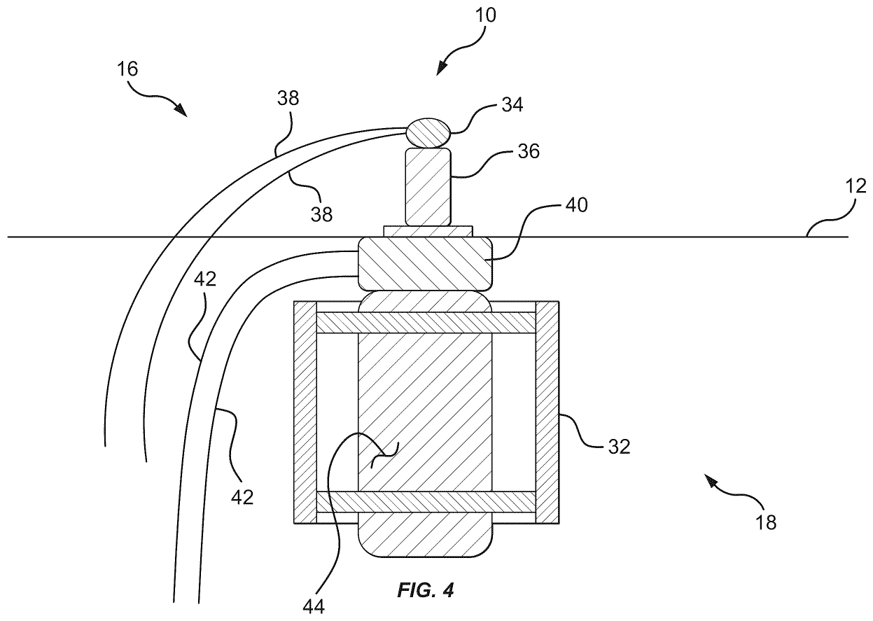

Referring now to FIGS. 3C and 4, the subject disposable sensor sub-assembly 16 is positioned internally of the mask 12 (in proximal relationship to the mouth and/or nose of the subject), and the non-disposable electronic sub-assembly 18 is preferably positioned externally of the mask 12.

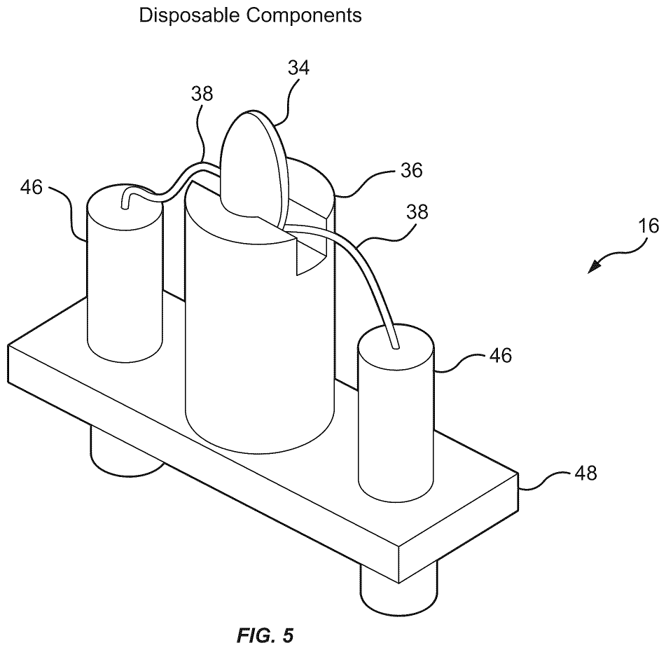

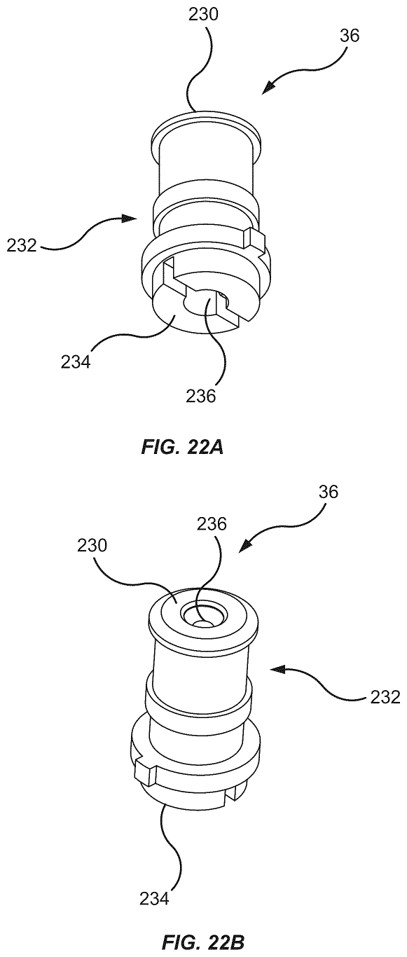

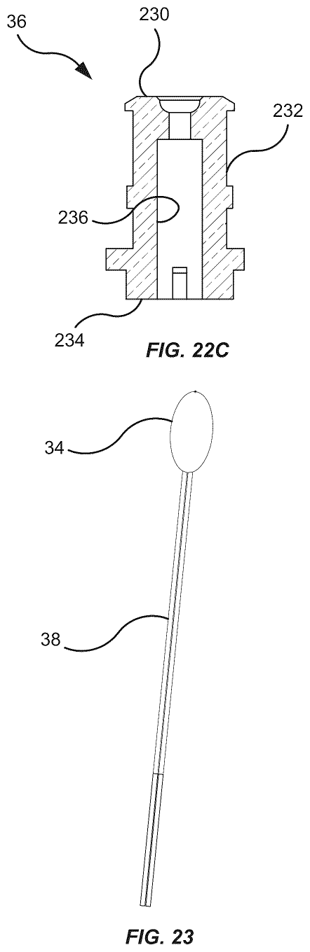

As presented in FIG. 4, the disposable sensor sub-system (sub-assembly) 16 and non-disposable electronic sub-system (sub-assembly) 18 are coupled together at two sides of the mask 12. FIG. 5 is representative of one of numerous embodiments of the present respiration monitoring system 10. Shown in FIGS. 4-5, in one of the exemplary embodiments, the system 10 includes a thermistor element 34 retained in place by a thermistor retainer 36 (detailed also in FIG. 22A-22C), also referred to herein as a thermistor mass OFC (Oxygen Free Copper). A pair of thermistor wires (leads) 38 extend in electrical contact with the thermistor element 34. In one of the examples, the thermistor leads 38 are formed as magnet wires 32 AWG. The thermistor leads 38 are connected to two contact pins 46. The contact pins 46, as shown in FIG. 5, extend through the alignment tool 48 which may be beneficial for alignment of the parts and portions of the system, as will be detailed in further paragraphs. The thermistor element 34 may be attached on the top of the brass column (also referred to herein as a thermistor hold or thermistor retainer) 36 with a conductive epoxy or any other adhesive component. The thermistor wires 38 of the thermistor element 34 are insulated with a very thin insulation. The non-disposable electronic sub-assembly 18 is configured with the thermal electric cooler (TEC) 40 with the leads 42 (for example, PTFE 28 AWG) extending therefrom. The TEC 40 is positioned in thermal contact with a heat sink 44 which may be formed from an OFC material. The TEC 40, leads (wires) 42, thermal sink 44 are encased in the non-disposable housing 32.

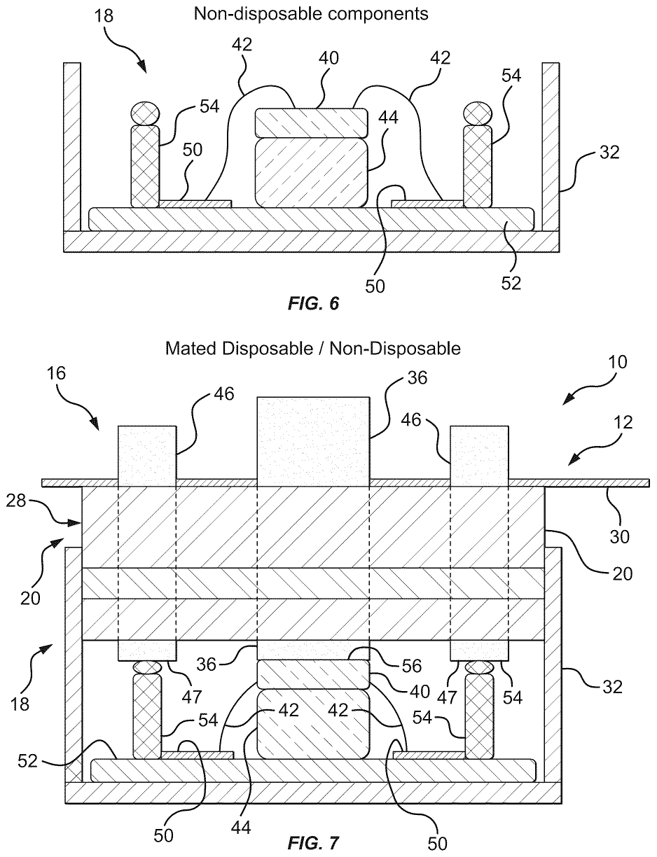

Referring to FIG. 6, which is a simplified representation of the non-disposable electronic sub-assembly 18, such is installed within the injection molded housing 32 supporting a Printed Circuit Board (PCB) 52 (which may be made from polyimide) carrying all necessary PCB connections/leads 50 created between the elements positioned on the PCB 52 and spring contacts 54, as well as all other non-disposable electronic components (and the disposable components of the present respiration monitoring system 10 when the disposable sub system 16 and non-disposable sub-system 18 are assembled together). The spring contacts 54 may be fabricated as MillMax 0900-0-15-20-76-11-0 type or any other spring contacts appropriate for the present system.

FIG. 7 shows, in a simplified format, the disposable sensor sub-system 16 and non-disposable electronic sub-system 18 mated to one another through a coupling (locking) mechanism 20 which includes a disposable sensor carrier 28. The sensor carrier 28 attaches the non-disposable housing module 32 (incorporating the non-disposable components) at one side of the wall 30 of the mask 12 to the disposable sensor sub-assembly 16 at another side of the wall 30 of the mask 12. As shown in FIG. 7, the connecting mechanism 20 is configured to connect, in a corresponding alignment, the bottom edges 47 of the contact pins 46 of the disposable sensor sub-assembly 16 with the spring contacts 52 of the non-disposable electronic sub-assembly 18, as well as a TEC 40 of the non-disposable electronic sub-assembly 18 with the bottom edge 56 of the probe (thermistor retainer) 36, so that the disposable components 16 are maintained in electrical/thermal/mechanical contact with the non-disposable components 18.

One of the key features of the present invention is to provide a locking mechanism between the disposable sensor sub-assembly 16 (directly attached to the mask 12 through the cut-out 22 formed in the wall 30 of the mask 12) and the non-disposable electronic sub-assembly 18. The locking mechanism 20 holds the disposable and non-disposable sub-systems in mechanical, electrical and thermal connection in a manner which would avoid any inadvertent dislodging from one another (when in use) and which would be able to easily separate from one another when the respiration monitoring is completed.

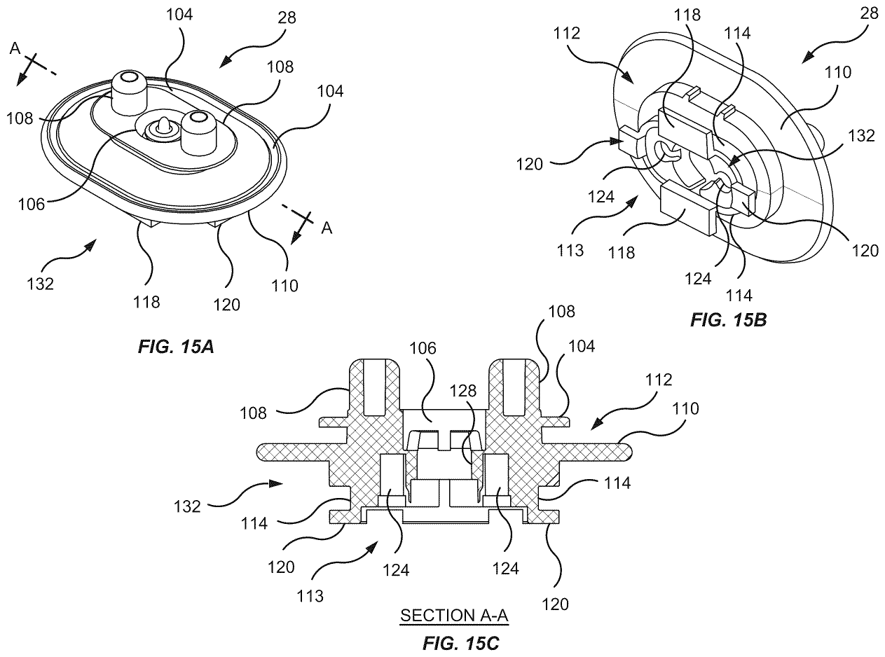

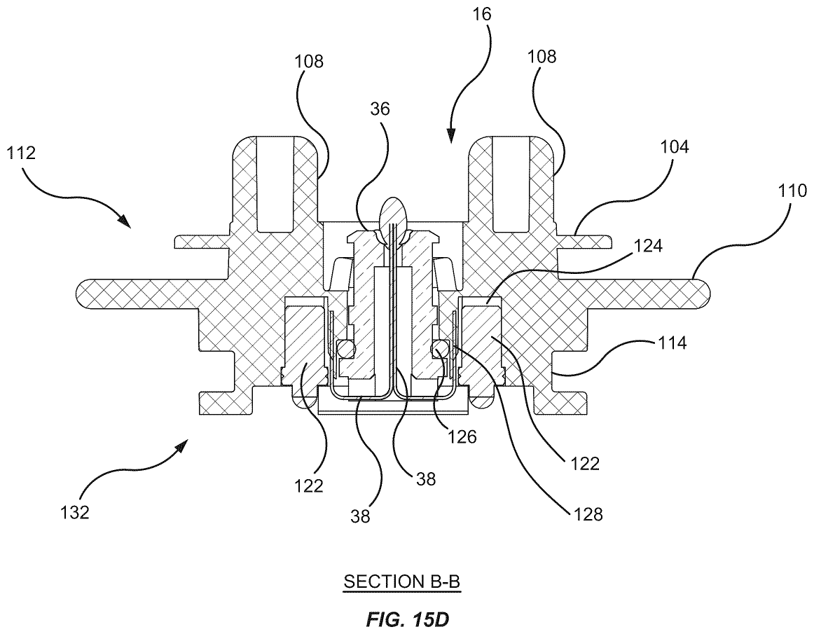

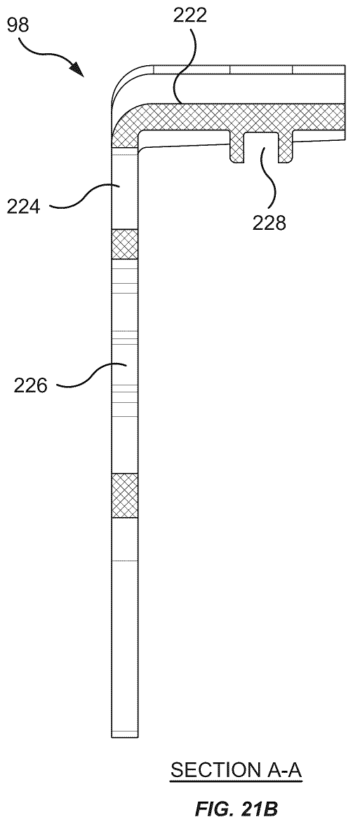

The locking mechanism 20 is specifically designed for electrical/thermal coupling/decoupling of the disposable and non-disposable components to/from one another. The locking sub-system 20 is designed with the intention that the disposable thermistor retainer 36 which holds the disposable thermistor element 34 attached thereto makes a sufficient pressurized contact with the cooling plate (TEC) 40 which is a part of the non-disposable electronic sub-assembly 18 (as shown in FIGS. 4 and 7). In the present system 10, a spring force is developed to push the disposable thermistor retainer 36 (which is fabricated from either brass or OFC material) against the cooling plate (TEC) 40 which, in its turn, also is connected to the PCB wires 50 arranged on the PCB sub-assembly 52 inside the housing 32 of the non-disposable sub-system 18. Such pressurized electrical coupling and thermal action between the disposable components 16 and non-disposable components 18 is provided, as will be detailed in the following paragraphs in conjunction with FIG. 12B, by the action of the non-disposable compression spring latch 98 (best shown in FIGS. 12B, and 21A-21B), as well as disposable POGO pins 122 (best shown in FIGS. 12B and 15D), as will be detailed in further paragraphs.

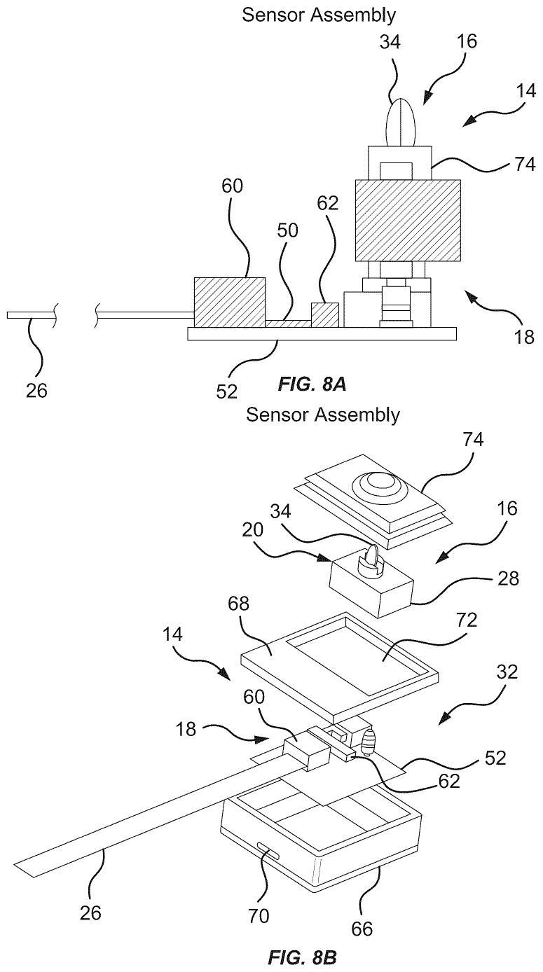

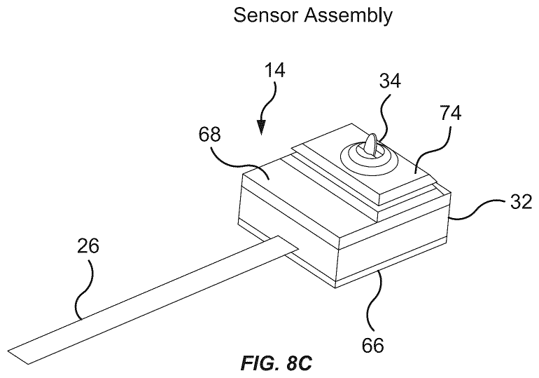

As presented in FIG. 8A-8C, showing an assembly of sensor components in one of its numerous embodiments, the PCB sub-assembly 52 has a cable landing pad 60 attached thereon, the TEC landing pad 62, and the mated disposable/non-disposable sub-assembly 14. The TEC leads are coupled to the TEC landing pad 62, and the sensor cable 26 is coupled to the cable landing pad 60. The PCB wires 50 interconnect the respective components to each other. The non-disposable electronic sub-system 18 is housed within the housing 32 with the PCB sub-assembly 52 supported by the rear portion 66 of the housing 32 and covered with the front portion 68 of the housing 32. The cable 26 enters through the opening 70 of the housing 32 into contact with the PCB 52 via connection with the cable landing pad 60.

The disposable sensor sub-system 16 is removably attached to the non-disposable electronic sub-system 18 through the connecting mechanism 20 which includes the sensor carrier 28 “stuffed” with the disposable components. As shown in FIG. 8B-8C, the thermistor element 34 extends through the front opening 72 formed in the front portion 68 of the housing 32. The front-most panel 74 has an opening for the thermistor element 34 to extend therethrough. The arrangement shown in FIG. 8A-8C is one of numerous exemplary arrangements and embodiments of the present system, and the scope of the present invention is not limited to this exemplary embodiment.

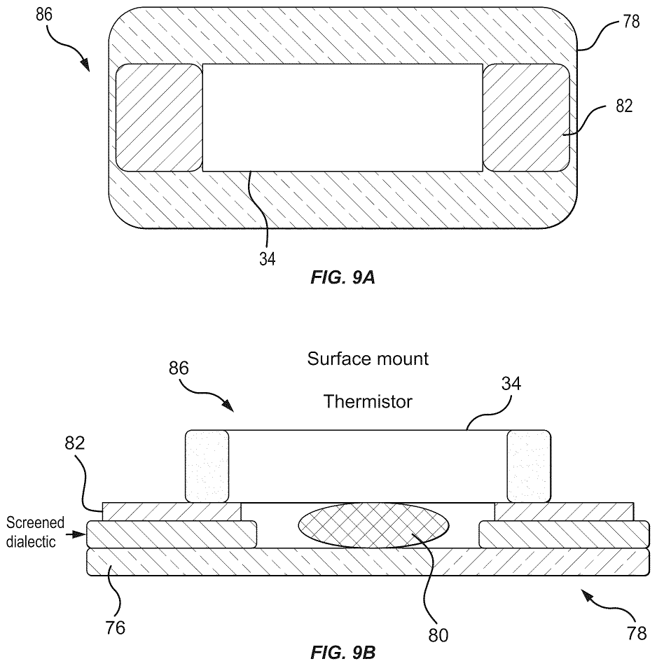

FIG. 9A-9B are representative of another alternative embodiment of the present system, i.e., including a surface mounted thermistor structure 86, where the thermistor element 34 is surface mounted on the contact pad 76 to attain a very low-profile structure with the overall height of the entire device of approximately 2 mm. FIG. 9B is a side view of the surface mounted thermistor design of FIG. 9A, where the thermistor element 34 is electrically coupled to the PCB 78 and is attached to the copper contact pad 76 by the thermally conductive epoxy 80. As shown in FIG. 9A-9B, the screened conductor pad and traces 82 are formed on the PCB 78, and the thermistor element 34 is in electrical connection thereto.

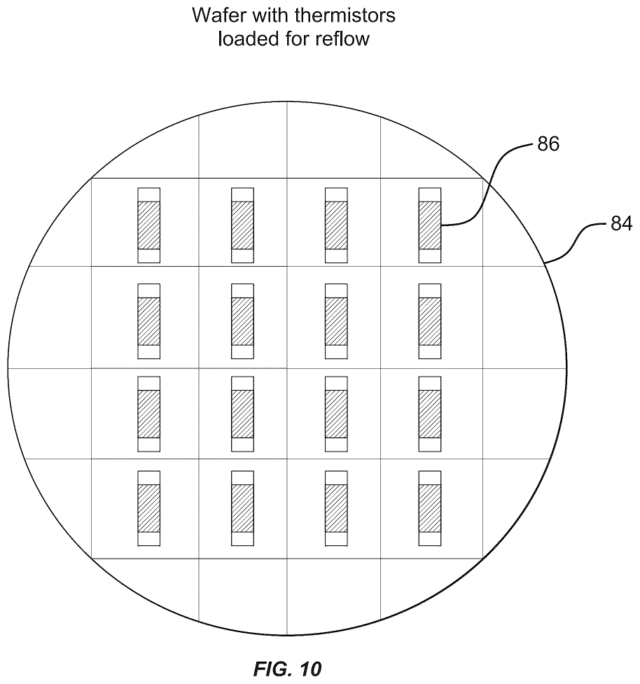

As shown in FIG. 10, the embodiment of the surface mounted thermistor 86 shown in FIG. 9A-9B may be fabricated in mass production. For such fabrication process, a wafer 84 is fabricated with a plurality (array) of the surface mounted thermistor structures 86 and loaded for the reflow process as it is known to those skilled in semiconductor production art.

Returning to FIG. 9A-9B, illustrating the surface mounted thermistor 86, such provides the subject system having a miniaturized profile. Instead of using the POGO pins, used for the spring pressurized electrical connection between disposable and non-disposable sub-assemblies 16, 18, there may be a different mechanism for providing thermal and electrical transfer between the disposable sensor sub-system 16 and non-disposable electronic sub-system 18. For example, instead of using the POGO pins, indentation may be formed on the conducting surfaces which may be gold coated to attain the function of raised spring surfaces which can be built either into the PCB itself (within the non-disposable electronic sub-system 18) or into the disposable sensor sub-system 16 to allow for the thermal and electrical coupling. This mechanism would benefit the production of the subject device when scalability and industrial volumes are of importance.

The process of fabrication of the surface mounted thermistors (allowing to produce hundreds of the disposable thermistors per wafer) may include the following operations:
start with a standard wafer size (6″ or 8″ in diameter, 0.5 mm thick) of copper, or thin layer of BECU, to form contact pad 76;
deposit dielectric or photoresist acrylic and cure (using heat or UV to form the screened dielectric);
deposit and cure a conductor material on the screened dielectric to form the screened conductor pad and traces 82;
place EpoxySet EB-3160C-2 thermally conductive drop 80 under the thermistor to keep in place the thermistor chip 86;
reflow the thermistor chip; and
using wafer saw, cutting individual disposable surface mounted thermistor structures 86.

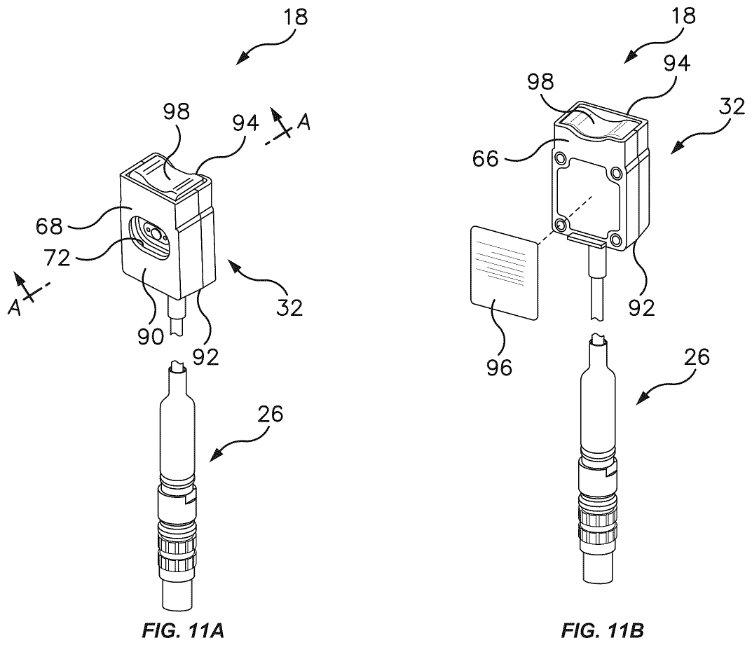

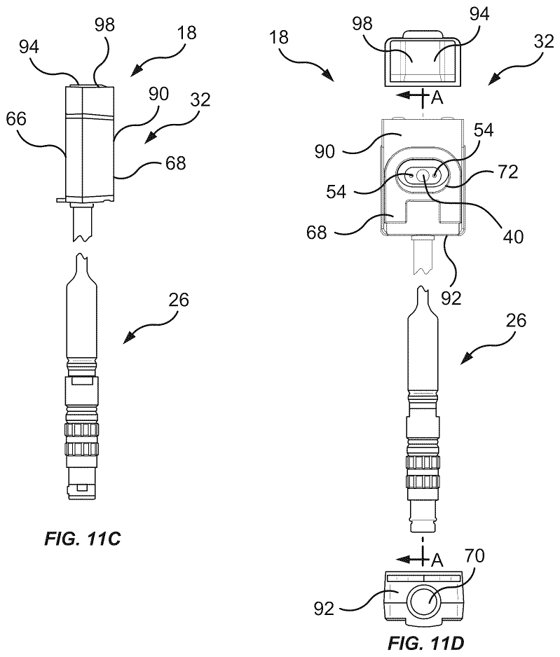

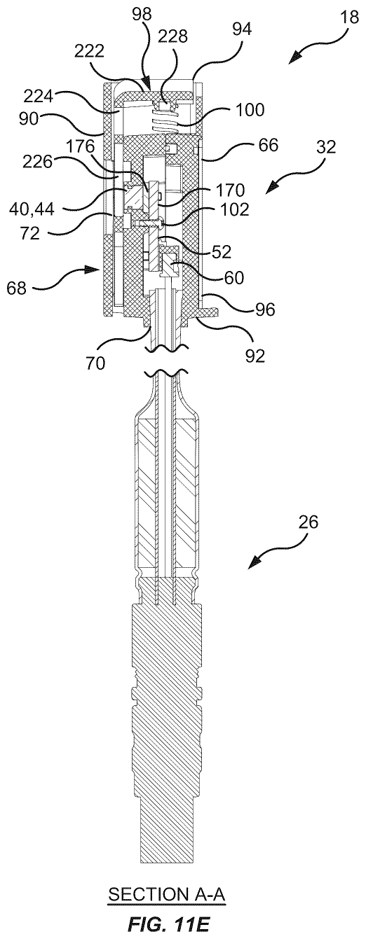

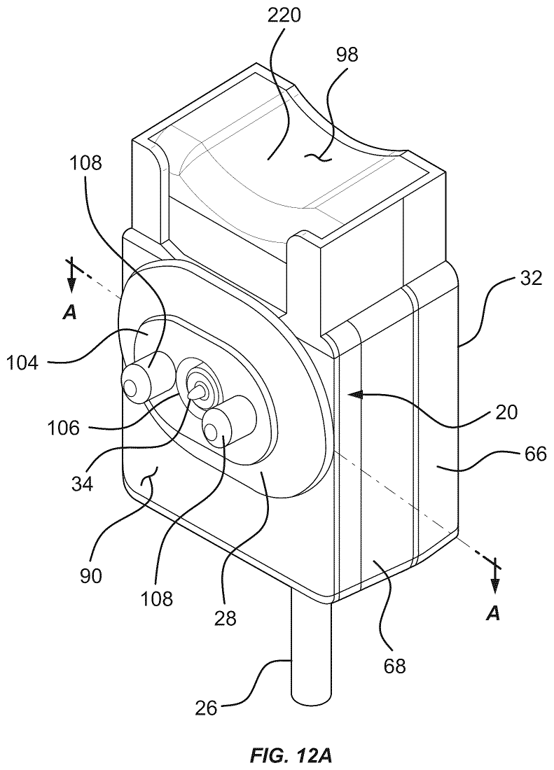

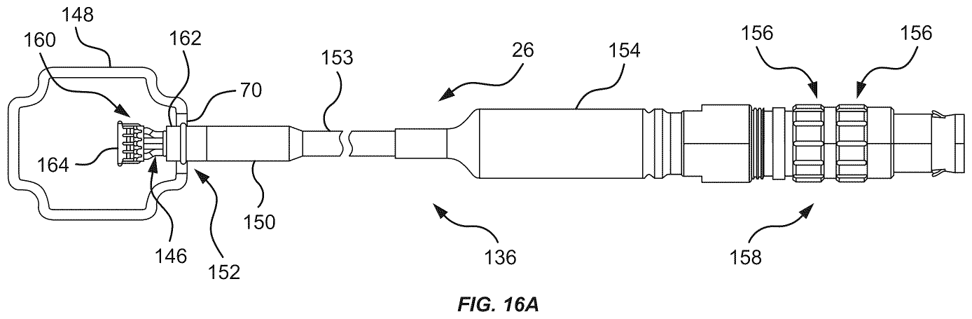

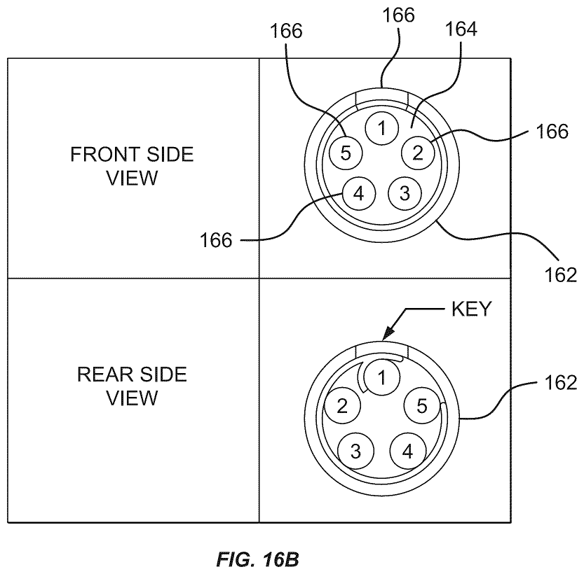

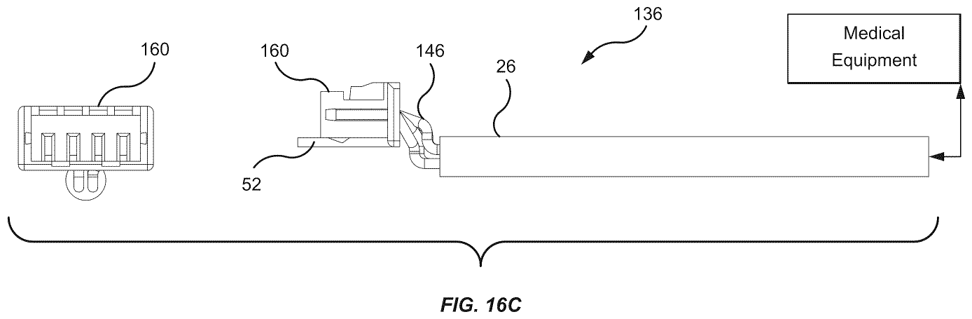

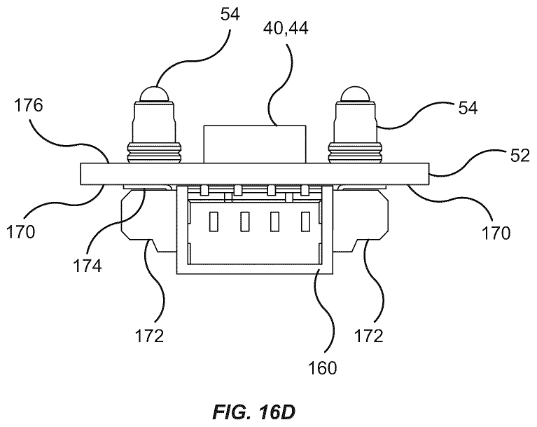

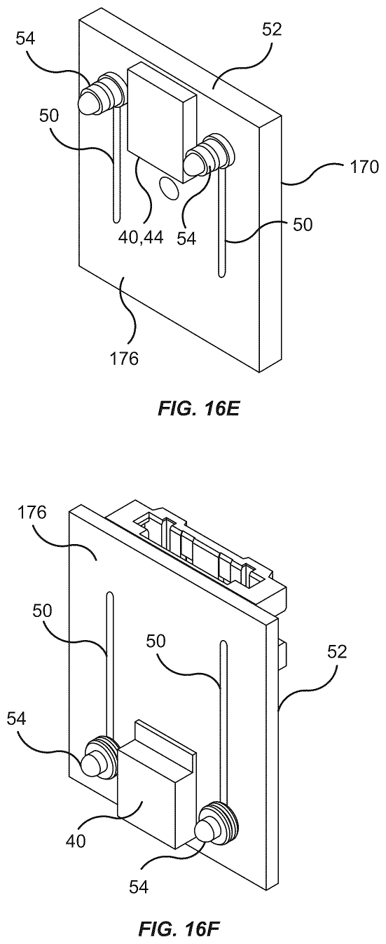

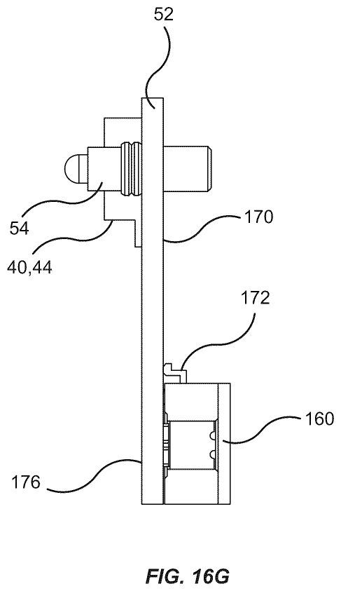

FIG. 11A-11E detail the non-disposable electronic sub-assembly 18 which includes the non-disposable housing (also referred to herein as a housing module) 32 encasing the non-disposable electronic components. The sensor cable 26 extends between the medical equipment (schematically shown in FIG. 16C) which processes and/or displays the respiration parameters measured by the system 10 and the non-disposable housing module 32, specifically, in an electric coupling with the electronic components (PCB 52, etc.) inside of the housing module 32.

As shown in FIGS. 11A-11E, the housing module 32 includes the front portion 68 and the rear portion 66 secured to one another via a system of connecting elements, such as, for example, plastic screws, pins, latches, magnets, adhesives, etc. As shown in FIGS. 11A and 11C-11E, the housing front portion 68 has a front surface 90 extending between the bottom wall 92 and the upper wall 94. The bottom wall 92 of the housing module 32 has the opening 70 (best shown in FIGS. 11D-11E) through which the sensor cable 26 is inserted into the housing module 32 for electrical connection with the non-disposable electronic components incorporated inside of the housing module 32. The cable 26 makes electrical connection with the electronics associated with the PCB sub-assembly 52 within the housing module 32. The rear portion 66 of the housing module 32 carries a label 96 glued to the housing 32.

A compression spring 100, best shown in FIG. 11E is installed within the housing 32 for properly positioning the non-disposable PCB sub-system within the housing 32. A compression spring latch 98 (best shown in FIG. 11A-11E and 21A-21B) is secured within the housing module 32 in proximity to the front portion 68 of the housing module 32 to facilitate proper positioning and attachment of the compression spring 100 within the housing 32 (best shown in FIG. 11E).

The front portion 68 of the non-disposable housing module 32 is formed with the front opening 72 which is configured to correspond to the contour of the sensor carrier 28 which is secured to the non-disposable housing module 32 at the front opening 72, as will be detailed in following paragraphs.

As shown in FIG. 11E, the PCB sub-assembly (also referred to herein as the PCB) 52 is secured inside the housing module 32 with a screw element 102 with a screw element 102 securing the PCB 52 to the internal plastic parts of the housing 32. The cable landing pad 60 is secured to the bottom surface 170 of the PCB 52, and the TEC 40 with the heat sink 44 attached to the opposite (top) surface 176 of the PCB 52. The TEC 40 and the spring contacts 54 are exposed within the front opening 72 formed in the front portion 68 of the housing module 32 for mating with the POGO pins 122 and the thermistor retainer 38 of the disposable sensor sub-assembly 16 when assembled to one another.

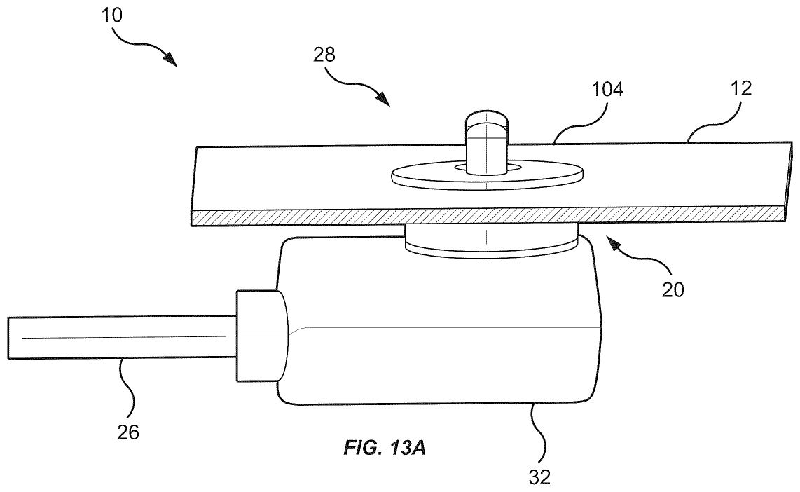

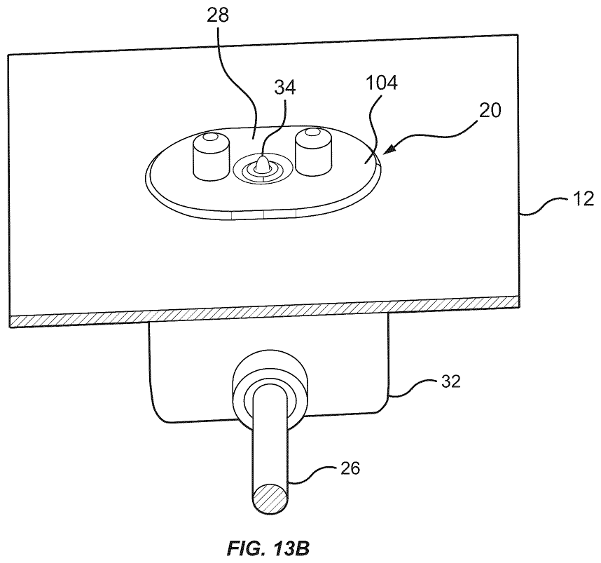

As shown in FIGS. 12A-12B, 13-A-13-B, 14A-14B, and 15A-15D, the disposable sensor carrier 28 is configured for attachment to the non-disposable housing module 32 and to the disposable mask 12. The disposable sensor carrier 26 constitutes a part of the subject connecting mechanism 20 and is used for securing the disposable sensor sub-assembly 16 to the non-disposable electronic sub-assembly 18 and to the disposable mask 12 in a manner shown in FIG. 13A-13B. The disposable sensor carrier 28 includes a plastic body having a top oval shaped plate 104 having a central opening 106 for exposing the thermistor element 34 to the internal atmosphere within the disposable mask 12. Two studs 108 are formed on the top oval plate 104 of the sensor carrier 28 at both sides of the central opening 106.

Vertically spaced apart from the top oval plate 104, the disposable sensor carrier 28 is formed with a lower oval shaped plate 110 which, as best shown in FIGS. 12B and 15A-15D) underlies the wall 30 of the disposable mask 12 while the plate 104 is disposed on the opposite side of the wall 30 of the mask 12, so that the wall 30 of the disposable mask 12 is sandwiched between the plates 104 and 110.

As best shown in FIGS. 12B and 15B-15D, the disposable sensor carrier 28 is configured with the outer surface 112 which forms indentations 114 configured for receiving a compressing spring latch 98 (as best shown in FIGS. 11E and 12B) for stabilizing the position of the sensor carrier 28 with regard to the housing 32 and to provide proper positioning of the electronics 18 within the housing 32 through the compression force of the spring 100. As shown in FIGS. 15B-15C, the indentations 114 are formed at the bottom 113 of the sensor carrier 28 by the tabs 118 and 120 which are formed for cooperation with the configuration of the front opening 72 of the front portion 68 of the housing module 32 to provide a reliable fixation of the non-disposable housing module 32 to the wall 30 of the mask 12 when in use, through the sensor carrier 28.

As detailed in FIGS. 12B, 14B and 15D, showing the cooperation between the disposable sensor components and the disposable sensor carrier 28, the disposable sub-assembly 16 includes the thermistor element 34 secured to the top of the thermistor retainer 36 and two POGO pins 122 inserted into the receptacles 124 configured in the body of the sensor carrier 28 (as best shown in FIGS. 15B-15C). The disposable sub-assembly 16 further includes a silicone O-ring 126 which is positioned in surrounding relationship with the thermistor retainer 36 and the internal wall 128 of the sensor carrier 28 to provide a reliable relative positioning and sealing of the components 16 within the disposable sensor carrier 28, one with respect to another.

Referring to FIG. 22A-22C, the thermistor retainer 36 includes a top portion 230 to which the thermistor is secured, a body 232 having a predetermined length, and a bottom portion 234. A central channel 236 extends inside the body 232 of the thermistor retainer 36 between the top portion 230 and the bottom portion 234. The thermistor 34 (shown in FIG. 23) is secured (preferably, through a heat transfer epoxy/adhesive) to the top portion 230 of the thermistor retainer 36. As shown in FIGS. 12B, 14B, and 15D, the thermistor wires 38, upon exiting from the channel 236 at the bottom portion 234 of the thermistor retainer 36, come into the compression-based contact with the BOGO pins 122, which further, in their turn, create the electrical contact between the thermistor leads 38 and the PCB wires 50 populating the PCB 52 through the spring contacts 54. The disposable sub-system 16 incorporated into the disposable sensor carrier 28 is thus brought in the mechanical/electrical stable and reliable connection to the non-disposable electronic sub-assembly 18. In addition to the mechanical/electrical, the thermal connection between the disposable and non-disposable sub-assemblies 16, 18 is also created when the bottom portion of the thermal retainer 36 is brought in contact with the TEC 40 of the non-disposable electronic sub-assembly 18 and stably maintained connected through the duration of the respiration monitoring cycle.

Referring again to FIGS. 12A-12B, 13A-13B, and 14A-14B, the disposable sensor sub-assembly 16 is positioned at the inside of the mask 12 and the non-disposable electronic sub-assembly 18 is positioned outside the mask 12, and are coupled to each other via the locking sub-system 20 which is the mechanism permitting to assemble the disposable sensor sub-assembly 16 and the non-disposable electronic sub-assembly 18 together in a quick but reliable manner.

Returning to FIGS. 4-7, there are two sets of wires used for electrical connection/disconnection of the disposable sub-system 16 and the non-disposable sub-system 18. The two sets of the wires in the subject system 10 include (a) a disposable set of the wires 38 of the thermistor element 34 embedded within the disposable sensor sub-system 16, and (b) the set of TEC wires 42 electrically coupled to the PCB leads (wires) 50 formed on the PCB 52 and other electrical components/electronics, which are embedded in the non-disposable sub-electronic assembly 18. In the disposable sensor sub-system 16, the thermistor wires 38 extend from the thermistor element 34 through the central channel 236 formed within the brass (or copper) column (also referred to herein as the thermistor retainer) 36. At the bottom 234 of the column 36, the thermistor wires 38 separate one from another and extend in opposite directions just beneath the disposable sensor carrier 28 into electrical contact with the POGO pins 122.

The POGO pin is a device that has a spring inside of the gold coated canister that has a smaller bobbin on the top. When the POGO pins are pressed on, a spring force develops inside the canister. The POGO pins 122 are made conductive to conduct electricity from the thermistor wires 38. The POGO pins 122 are installed in the disposable sensor carrier 28 to press down on the connection pins 130 (best shown in FIG. 12B) of the non-disposable electronic sub-assembly 18. The connection pins 130 are positioned between the POGO pins 122 and the spring contacts 54 on the PCB 52, and thus, when the POGO pins 122 (of the disposable sensor sub-system 16) press down on the connection pins 130 (of the non-disposable electronic sub-system 18), an electrical connection between two sets of the wires, i.e., the thermistor wire 38 and the TEC wires 42 and the PCB connectors 50 is attained and maintained reliably during the respiration monitoring cycle, as needed.

When the subject system 10 is in use, the disposable sub-system 16 is in coupling (electrical, mechanical, and thermal) with a non-disposable sub-system 18 and the POGO pins 122 are pressed down to make contact between the thermistor wires 38 and the TEC wires 42, as well as the PCB wires 50. Once the sub-system 16 is disconnected from the sub-system 18, the POGO pins 122 are released, and the disposable sub-system 16 (including the thermistor 34, thermistor wires 38, and the thermistor retainer 36) along with the mask 12 can be discarded, while the non-disposable electronic components can be kept for further re-use with another mask 12 for another subject.

In the present system, the thermistor retainer 36 of the disposable sensor sub-system 16 provide a reliable contact with the TEC 40 of the non-disposable electronic sub-system 18, as well as with the electronics underneath. As an example, such contact may be provided through the arrangement containing the POGO pins 122 and the compression spring latch 98 positioned in contact with the disposable sensor carrier 28, as shown in FIG. 12B. The present system 10 also includes the O-ring 126 shown at least in FIG. 12B, which is subjected to the compression force when the device 10 is assembled. This allows a few ounces of force to press the brass (or copper) thermistor retainer 36 against the cooling plate (TEC) 40 positioned in thermal contact with the bottom 234 of the column 36. Thus, as one of the distinct features of the present system 10, in addition to coupling/decoupling the disposable sub-system 16 and the non-disposable electronic sub-system 18, the locking mechanism 20 which is operatively coupled therebetween, also provides transfer of the thermal energy between the disposable sensor sub-system 16 and non-disposable electronic sub-system 18, when coupled together.

As shown in FIGS. 12B, 14A-14B and 15A-15D, the locking mechanism 20 includes an interface 132 between the disposable sensor carrier 28 and the non-disposable housing module 32. The interface 132 is provided by forming the mating configurations of the outer surface 112 and the bottom surface 132 of the disposable sensor carrier 28 and mating surfaces of the front portion 68 of the non-disposable housing module 32. The lower plate 110 of the disposable sensor carrier 28, when is brought in mating position with the housing module 32 of the non-disposable sub-assembly 18 (as shown in FIG. 12B), comes in contact with the front portion 68 of the housing module 32. Also, the outer surface 112 of the disposable sensor carrier 28 is configured with indentations 114 which accommodate the compression spring latch 98 to facilitate the coupling to the front portion 68 of the housing 32 through the latch 98 which fastens the mating portions of the sensor carrier 28 and the indented portions 136 of the front portion 68 of the housing module 32. The tab 118, 130 on the lowest portion 113 of the sensor carrier 28 are engaged with the receptacles 140 of the front portion 68 of the housing module 32. Since the front portion 68 of the housing module 32 and the disposable sensor carrier 28 are formed from a plastic, the interconnections between the mating portions of the interface configuration therebetween are flexibly and resiliently assembled/disassembled as required by operation of the subject system 10. The PCB 52 is received within the housing 32 between its front portion 68 and the rear portion 66. The PCB 52 has an array of electrical connections 50 deposited thereon which are electrically connected to the wires 146 of the sensor cable 36.

FIGS. 16A-16G depict the sensor cable assembly 136 which may, in one of the embodiments, be fabricated as a multi-conductor cable 26 having four wires 146. As best shown in FIGS. 16A and 16C, the wires 146 of the sensor cable 26 enter the housing 32 through the gasket 148 (best depicted in FIGS. 12B, 16A and 20) which is sandwiched between the front portion 68 and the rear portion 66 of the housing module 32. The gasket 148 is formed with a cable strain relief member 150 heat-shrunk around the end 152 of the sensor cable 26 to ease the strain formed between the sensor cable 26 and the housing module 32. The sensor cable 26 includes an internal sheath 153 passing longitudinally within the external sheath 154, which can be telescopically disposed one with respect to another through the push-pull manipulators 156 at the end 158 of the sensor cable 26.

At the end 152 of the sensor cable 26, the wires 146 are coupled to the cable connector housing 160 (similar to the cable landing pad of FIGS. 8A-8C). At the bottom wall of the housing module 32, at the interconnection between the front portion 68 and rear portion 66 of the housing module 32, a rounded passage 162 is formed which is aligned with the strain relief member 150 in the gasket 148. The rounded passage 162 is provided with a wall 164 configured with openings 166, each opening 166 for passing a pin of the respective wire 146. For example, as shown in FIG. 16B, the openings 166 (1 and 2) are provided for pins of the TEC wires (plus) and TEC wires (minus). The openings 166 (3 and 4) are provided for passing pins for the thermistor wires (plus) and thermistor wires (minus). The opening 166 (5) is for termination of the shielding of the cable 26.

Referring to FIG. 16C-16G, the cable connector housing 160 is attached to the bottom surface 170 of the PCB 52. The cable connector housing 160 is connected to the bottom surface 170 of the PCB 52 mechanically (for example, by soldering) and electrically via the gold-plated contacts 172. The contacts 172, as shown in FIGS. 16D and 16G, extend between the cable connector housing 160, specifically, between the cable wires 146 of the sensor cable 26 to the bottom electrical pads 174 formed on the bottom surface 170 of the PCB 52 and in electrical contact with spring loaded contact connectors (spring contacts) 54 of the non-disposable sub-assembly 18 of the subject system 10. The spring-loaded contacts 54 extend on the top surface 176 of the PCB 52 and are brought in mechanical and electrical contact with the contact pins 46 of the disposable sub-assembly 16, when the system 10 is assembled.

In addition to the spring contacts 54, the top surface 176 of PCB 52 carries the TEC 40 and the heat sink 44 as shown in FIGS. 4 and 6-7, and as disclosed in previous paragraphs.

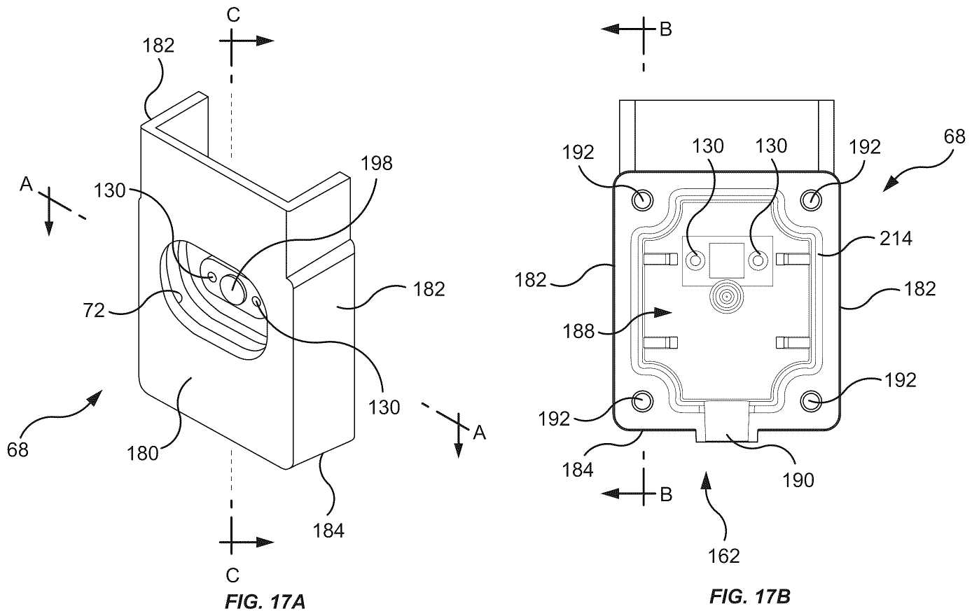

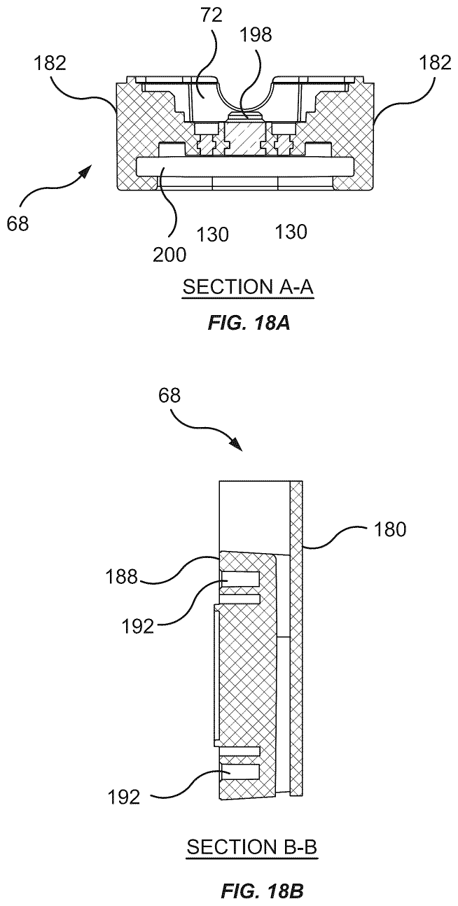

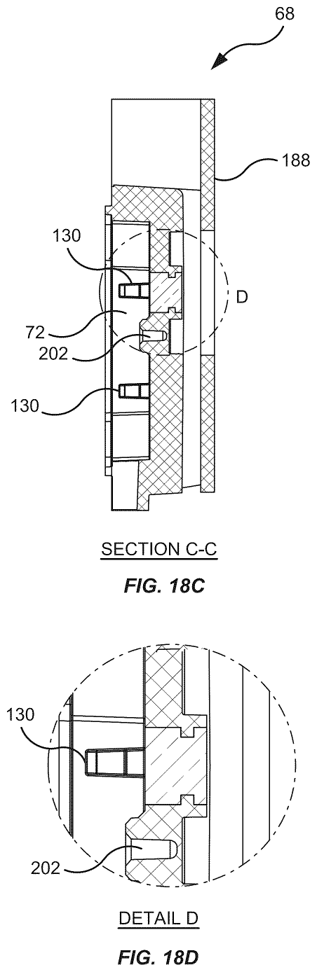

Referring to FIG. 17A-17B and 18A-18D, the housing module 32 includes a front portion 68 which has a front panel 180, two sidewalls 182, and a bottom wall 184. The front panel 180 has a cutout 72 formed therein and configured in a contour corresponding to the oval contour of the sensor carrier 28. The rear side 188 of the front panel 180 is shown in FIG. 17B and includes a portion 190 of the rounded passage 162 which is formed in the bottom wall 184 of the front portion 68 of the housing module 32, for passing the end 152 of the sensor cable 26. The gasket 148 is positioned in attachment with the rear side (surface) 188 of the front panel 180.

Referring to FIG. 18A, which is a cross-section taken along Lines A-A of FIG. 17A, the front portion 68 is configured with the connection block 198, and a pair of connection pins 130 which are brought in contact with the contact pins 46 of the disposable sensor sub-assembly 16 and the spring contacts 54 of the non-disposable electronic sub-assembly 18, and specifically, with the PCB wires 50 on the PCB 52 which is positioned in the PCB receptacle 200 in the housing module 32.

As shown in FIGS. 17B and 18B, four corner indentations 192 are provided for passing connecting pins which would be aligned with the same indentations formed in the rear portion 66 of the housing 32, as will be detailed in further paragraphs. Two connection pins 130 extend through the front panel 180.

Referring to FIG. 18C, which is a cross-section of FIG. 17A taken along Lines C-C, a channel 202 is formed to pass a pin through to connect the connection block 198 with the rear surface 188 of the housing panel 180. FIG. 18D shows the detail D of FIG. 18C.

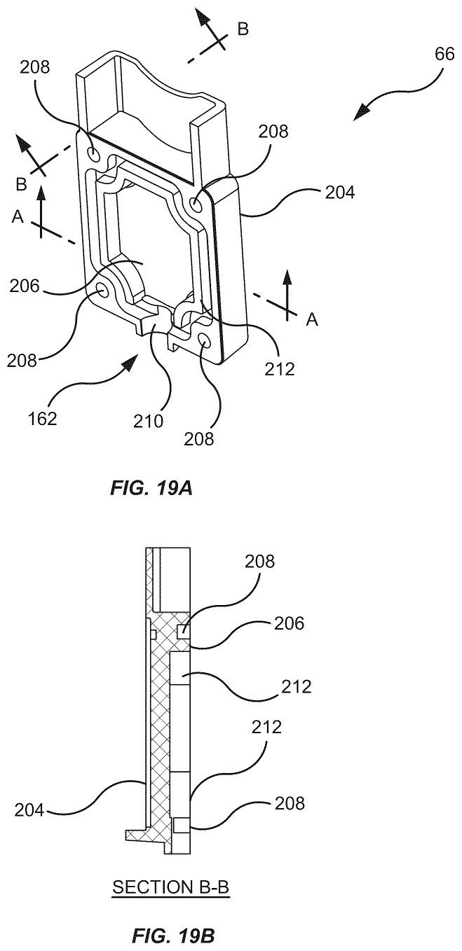

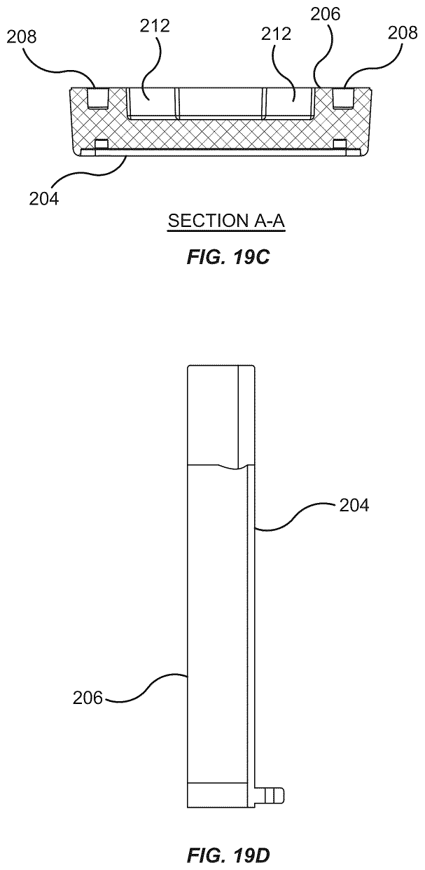

FIG. 19A-19D depict the rear portion 66 of the non-disposable housing 32 which includes an external surface 204 and an internal surface 206. The internal surface 206 is configured with four corner channels 208 for connection with the four corner indentations 192 of the front portion 68 of the housing module 32. The connection between the corner channels 208 and 192 can be through soldering, through adhesives, or through pins inserted in the corner channels 208 and the corner indentation 192 and coupling of the front portion 68 and the rear portion 66 of the non-disposable housing module 32 to each other.

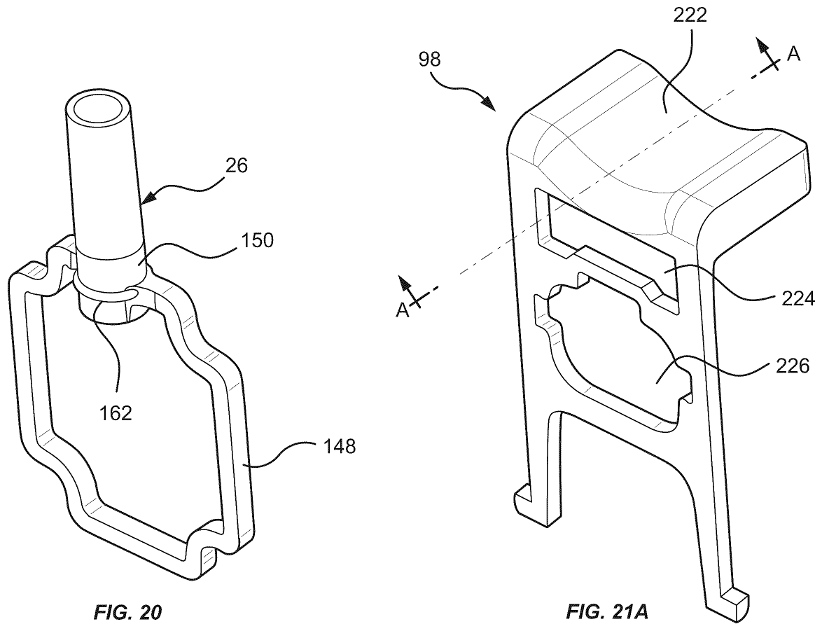

A portion 210 and the portion 190 formed in the front portion 68 of the housing 32 together form the rounded passage 162 for entering the wires 146 of the cable 26 into the cable connector housing 160. The gasket 148 is inserted into the gasket channel 212 which is formed in alignment with the gasket channel 214 at the front portion 68 of the housing 32. FIG. 20 details the configuration of the gasket 148 configured with the rounded passage 162 for entering the sensor cable 26 into the non-disposable housing module 32.

Referring to FIGS. 21A-21B taken in conjunction with FIGS. 11E and 12A-12B, the non-disposable housing module 32 can be configured with the compression spring latch member 98 which is incorporated into the housing module 32, as depicted in FIG. 11E, to cooperate with the coil spring 100. The compression spring latch member 98 is configured with an opening 224 aligned with the compression spring 100, and an opening 226 configured in alignment with the sensor carrier 28 when assembled together. As shown in FIGS. 11E and 21B, the top 222 of the compression spring latch member 220 is provided with a spring holder channel 228 to which one end of the spring 100 is secured to provide proper positioning and alignment of the electronic components within the housing module 32.

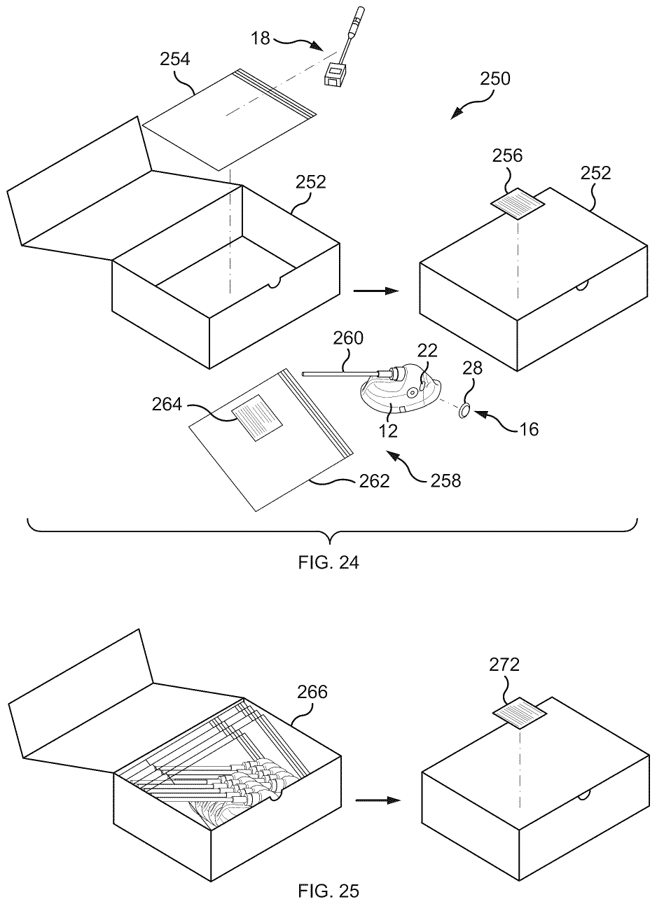

Referring to FIG. 24, a packaged assembly 250 is shown which includes a corrugated box 252, a clear zipper bag 254, a non-disposable sub-assembly module 18 which is inserted into the clear zipper bag 254 and may be packed (for example, in groups of 10, or 50, 100, etc., modules) into the corrugated box 252. A label 256 identifying the box containing non-disposable sensor modules is attached to the box 252.

Referring to FIG. 25, showing the packaged assembly containing ten disposable sensors/masks is shown where the disposable mask 12 configured with the cutout 22, along with the disposable sensor carrier 28 “stuffed” with the disposable components 16, is inserted in showing the connection to the respiratory system 260 is inserted into the clear zipper bag 262 carrying a label 264 indicating the disposable mask 12 and the sub-assembly 16 contained therewithin. A corrugated box 266 is packed with a specific number of plastic bags 262, each containing a disposable mask 12 with the disposable sensor sub-assembly 16, and the label 272 indicating that the box 266 contains a specific number of masks and disposable sensors 16 is attached to each box 266.

As presented in previous paragraphs, the front portion 68 and the rear portion 66 of the non-disposable housing module 32 of the non-disposable electronic sub-assembly 18 may be connected one to another by a system of screws with the gasket 148 positioned therebetween, and the PCB assembly 52 is positioned between the front 68 and rear 66 portions of the housing 32. The sensor cable 26 has the wires 146 inserted in electrical connection with the PCB assembly 52 through the rounded passage 162 formed in the housing module 32, as presented in previous paragraphs. The POGO pins 122 (as an alternative to the contact pins 46) of the disposable sensor sub-assembly 16, provide electrical connection between the thermistor leads 38 through the spring contacts 54 of the non-disposable sub-assembly 18 to the PCB assembly 52. As presented in previous paragraphs, the disposable sensor sub-system 16 is formed with the disposable sensor carrier 28 and the brass column (also referred to herein as thermistor retainer or holder) 36, to which the thermistor component 34 is attached via epoxy component or other adhesive arrangement.

In order to assemble the disposable sensor sub-assembly 16 to the non-disposable electronic sub-assembly 18, first the thermistor retainer 36 with the thermistor component 34 attached thereto is pressed into the disposable sensor carrier 28, and second, the sensor carrier 28 snaps in place in connection with the non-disposable sub-assembly 18 in the manner presented in previous paragraphs. While it is snapping into the place, the disposable sensor carrier 28 is under the load with the O-ring 126 that extends at the bottom 234 of the thermistor retainer 36 in surrounding relationship therewith. When the thermistor retainer 36 is snapped into the disposable sensor carrier 28, the O-ring is compressed, and provides locking function. Subsequently, the POGO pins 122 are pressed down to make a contact between the disposable thermistor wires 38 and the non-disposable PCB wires 50.

The thermistor component 34 is electrically coupled to the PCB assembly 52 (and/or other electric components/electronics in the non-disposable electronic sub-assembly 18) through two sets of wires, including the thermistor wire 38 extending in the disposable sensor sub-system 16, and the TEC wires 42 running in the non-disposable electronic sub-system 18.

The wires 38 coming from the thermistor component 34 through the central channel 236 configured within the thermistor retainer 36, are basically the conductors for measuring the thermistor's readings. At the bottom 234 of the thermistor retainer 36, the wires 38 are split and extend in opposite directions into contact with the POGO pins 122 on both sides of the thermistor retainer 36, and subsequently with the PCB connections 50.

Both pairs of the wires (thermistor wires 38 and the non-disposable sub-system's wires 42) are connected and disconnected by means of the POGO pins 122 when the disposable sub-system 16 and non-disposable sub-system 18 are snapped together or decoupled from one another, respectively. When the assembly 10 is disconnected into the disposable sensor sub-assembly 16 and the non-disposable electronic sub-assembly 18, the thermistor component's wires 38 and the non-disposable TEC wires 42 are disconnected from one another accordingly, so that the thermistor's wires 38 are disconnected from the TEC wires and the electrical part (PCB wires, etc.) of the sensor assembly 10.

There are numerous ways of providing reliable locking/mating mechanism between the disposable and non-disposable sub-systems in the present assembly 10, along with the assembled/disassembled modes of operations for thermal and electrical transfer between the disposable and non-disposable sub-systems 16, 18. Though described herein are merely the exemplary arrangements, and numerous alternative ways are contemplated in the present system for providing the subject locking mechanism capable of the electrical/thermal transfer between the disposable and non-disposable parts of the present system 10 which would allow to transfer the thermal control to the thermistor component into the disposable sub-system.

What is claimed is:

1. Portable respiration monitoring system, comprising:
a sensor/electronic assembly positionable in a respiration pathway by a positioning device, said sensor assembly being configured with
(a) a disposable sensor sub-assembly,
(b) a non-disposable electronic sub-assembly, and
(c) a mating sub-system operationally coupled to said disposable sensor sub-assembly and non-disposable electronic sub-assembly, said mating sub-system being configured for intermittent coupling and decoupling of said non-disposable sensor sub-assembly to/from said disposable electronic sub-assembly and said positioning device.

2. The system of claim 1, wherein said disposable sensor sub-assembly includes a thermistor element, a thermistor retainer configured to hold the thermistor element, a pair contact pins, and a pair of thermistor wires coupled between said thermistor element and said at least a pair of contact pins, and
wherein said mating sub-system includes a disposable sensor carrier housing incorporating said thermistor element, thermistor retainer, of transistor wires, and pair of contact pins, wherein said disposable sensor carrier housing is removably attached to said positioning device and to said non-disposable electronic sub-assembly.

3. The system of claim 2, wherein said non-disposable electronic sub-assembly includes:
a non-disposable housing module incorporating a Printed Circuit Board (PCB) sub-assembly carrying PCB connections thereon, a pair of spring contacts in electrical contact with said PCB connections, a thermal electric cooler (TEC), a pair of TEC wires coupled between said TEC and said PCB connections, and a heat sink in thermal contact with said TEC.

4. The system of claim 3, wherein said disposable sensor carrier is removably attached to said non-disposable housing module of said non-disposable electronic sub-assembly with said pair of thermistor wires brought into electric contact with said PCB connections via said pair of contact pins of said disposable sensor sub-assembly and said pair of spring contacts of said non-disposable electronic sub-assembly.

5. The system of claim 4, wherein said disposable sensor carrier is configured with two indentations, and wherein said pair of contact pins of said disposable sensor sub-assembly are configured as POGO pins, each POGO pin inserted in a respective one of said two indentations.

6. The system of claim 5, wherein said thermistor retainer has a retainer body formed of thermal conductive material and configured with a retainer top portion carrying said thermistor element, a retainer bottom portion positioned in contact with said TEC, and a retainer channel extending between said retainer top and bottom portions, and wherein said pair of thermistor wires extend within said retainer channel from said thermistor element out of said retainer body and further pressed down by said POGO pins to said PCB connections.

7. The system of claim 6, wherein said positioning device is a mask configured with a sensor cut-out formed in a wall of said mask, and wherein said disposable sensor carrier is attached to said wall of said mask via said sensor cut-out, with said thermistor element disposed inside of said mask.

8. The system of claim 7, wherein said non-disposable electronic sub-assembly further comprises:
a sensor cable having a wire sub-system extending therethrough, wherein said wire sub-system is positioned in electrical contact with said PCB connections.

9. The system of claim 8, wherein said non-disposable housing module includes a front portion, a rear portion attached to said front portion, a gasket member secured between said front and rear portions, and a cable entrance opening formed in a bottom wall of said non-disposable housing module for passing an end of said sensor cable therethrough into a contact with said PCB sub-assembly.

10. The system of claim 9, wherein said disposable sensor carrier includes two parallel plates displaced from one another to sandwich said wall of said mask therebetween.

11. The system of claim 10, wherein said mating sub-system is further configured with a disposable sensor carrier - non-disposable housing module interface, wherein said interface has a plurality of tabs formed at a bottom of the disposable sensor carrier, wherein the front portion of the non-disposable housing module is configured with a front wall having a front opening, and wherein said plurality of tabs cooperate with the front wall of the front portion of the non-disposable housing module within said front opening to connect said disposable sensor carrier and said non-disposable housing module to one another.

12. The system of claim 11, wherein said spring contacts of said non-disposable electronic sub-assembly extend within said front opening of said front wall of said front portion of the non-disposable housing module in electrical contact with said POGO pins and thermistor wires of said disposable sensor sub-assembly.

13. The system of claim 12, wherein said rear portion of said non-disposable housing module is secured to said front portion of said non-disposable housing module via a system of connecting pins.

14. The system of claim 12, wherein the PCB assembly is disposed in the rear portion of said non-disposable housing module.

15. The system of claim 9, wherein said non-disposable housing module further includes a compression spring disposed at top portion thereof to maintain the PCB assembly at a predetermined position within the non-disposable housing module.

16. The system of claim 15, wherein said non-disposable housing module further includes a compression spring latch positioned in proximity to said front portion of the non-disposable housing module and securing said compression spring in place.

17. The system of claim 1, further comprising a package including a plurality of said non-disposable electronic sub-assemblies packaged in a first corrugated box, with each said non-disposable electronic sub-assembly packaged in a plastic bag, and a plurality of said disposable sensor sub-assemblies and a plurality of said poisoning devices packaged in a second corrugated box, with each said disposable sensor sub-assembly and said positioning device packaged in a plastic bag.

18. A method for respiration monitoring system, comprising:
configuring a sensor/electronic assembly with a disposable sensor sub-assembly and a non-disposable electronic sub-assembly,
intermittently coupling said disposable sensor sub-assembly to a positioning device and to said non-disposable electronic sub-assembly for positioning said disposable sensor sub-assembly in a respiration pathway and operating said sensor/electronic assembly in a respiration monitoring mode of operation, and
decoupling said disposable sensor sub-assembly from said non-disposable electronic sub-assembly and from said positioning device upon completion of the respiration monitoring mode of operation.

19. The method of claim 18, further comprising:
disposing of said disposable sensor sub-assembly and said positioning device, and
reusing said non-disposable electronic sub-assembly.

20. The method of claim 18, further comprising:
configuring said disposable sensor sub-assembly with a disposable sensor carrier housing incorporating a thermistor element, a thermistor retainer configured to hold the thermistor element, a pair contact pins, and a pair of thermistor wires coupled between said thermistor element and said at least a pair of contact pins,
configuring said non-disposable electronic sub-assembly with a non-disposable housing module incorporating a Printed Circuit Board (PCB) sub-assembly carrying PCB connections thereon, a pair of spring contacts in electrical contact with said PCB connections, a thermal electric cooler (TEC), a pair of TEC wires coupled between said TEC and said PCB connections, and a heat sink in thermal contact with said TEC, and
in said respiration monitoring mode of operation, attaching said disposable sensor carrier to said positioning device and to said non-disposable housing module of said non-disposable electronic sub-assembly with said pair of thermistor wires brought into electric contact with said PCB connections via said pair of contact pins of said disposable sensor sub-assembly and said pair of spring contacts of said non-disposable electronic sub-assembly.
